SEQUENCE LISTING (14 sequences)

SEQ ID NO: 1 - DNA, 342 bp, Mus sp.
gtccaactgcagcagcctggggctgagcttgtgaagcctggggcttcagtgaagctgtcctgcaaggcttctggctacaccttcaccagctactggatgcactgggtgaagcagaggcctggacgaggccttgagtggattggaaggattgatcctaatagtggtggtactaagtacaatgagaagttcaagagcaaggccacactgactgtagacaaaccctccagcacagcctacatgcagctcagcagcctgacatctgaggactctgcggtctattattgtgcaagagggtactatggtatccactttgactactggggccaaggcaccactctcaca

SEQ ID NO: 2 - DNA, 597 bp, Homo sapiens
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 3 - DNA, 20 bp, Artificial
  primer
ttacctgggtctatggcagt

SEQ ID NO: 4 - DNA, 19 bp, Artificial
  primer
tgaaggctcagaatccccc

SEQ ID NO: 5 - DNA, 23 bp, Artificial
  primer
cccctcctttgccgacatcttcc

SEQ ID NO: 6 - DNA, 22 bp, Artificial
  primer
tccattcctcctcgtcacagtc

SEQ ID NO: 7 - DNA, 22 bp, Artificial
  primer
cacacagctcagacgcaacccc

SEQ ID NO: 8 - DNA, 28 bp, Artificial
  primer
ggatcatttaccaggagagtgggagagg

SEQ ID NO: 9 - DNA, 20 bp, Artificial
  primer
gaggcaagaagacactctgg

SEQ ID NO: 10 - DNA, 20 bp, Artificial
  primer
gtgacattcctggaagttgc

SEQ ID NO: 11 - DNA, 21 bp, Artificial
  primer
ccgctcttgccaaatgctttg

SEQ ID NO: 12 - DNA, 22 bp, Artificial
  primer
cacgatacttcatctcatctgg

SEQ ID NO: 13 - DNA, 21 bp, Artificial
  primer
agaaaatggtaggcgcgcgta

SEQ ID NO: 14 - DNA, 22 bp, Artificial
  primer
tcgactcatctcagcattaaag

(19) United States
(12) Patent Application Publication
(10) Pub. No.: US 2005/0095712 A1
(43) Pub. Date: May 5, 2005

(54) Mutations caused by activation-induced cytidine deaminase

(76) Inventors: Alberto Martin, Toronto (CA); Matthew D. Scharff, Larchmont, NY (US)

Correspondence Address: AMSTER, ROTHSTEIN & EBENSTEIN LLP, 90 PARK AVENUE, NEW YORK, NY, 10016, US

(21) Appl. No.: 10/501,628

(22) PCT Filed: Jan. 15, 2003
(86) PCT No.: PCT/US03/01149

Related U.S. Application Data
(60) Provisional application No. 60/350,269, filed on Jan. 17, 2002.

(51) Int. Cl. C12N 15/85

(52) U.S. Cl. 435/455

(57) ABSTRACT

Methods for causing mutations in genes expressed in eukaryotic cells are provided. The methods involve expressing an activation-induced cytidine deaminase (AID) in the cells. The mutated genes can be any gene that is operably linked to a promoter, where the gene is within about 2 kilobases of the promoter. Examples include antibody genes. Also provided are cells expressing AID. The cells can be from any eukaryote, and include hybridoma cells and myeloma fusion partners.

### BACKGROUND

(1) Field of the Invention

The present invention generally relates to methods and compositions for inducing mutations in genes in living cells. More specifically, the invention relates to the use of activation-induced cytidine deaminase to induce mutations in genes expressed in eukaryotic cells.

(2) Description of the Related Art

### REFERENCES CITED

Albanese, C. et al. FASEB J. 14, 877-884 (2000).
Arakawa, H., Hauschild, J. & Buerstedde, J. M. Science 295, 1301-6 (2002).
Bachl, J., Carlson, C., Gray-Schopfer, V., Dessing, M. & Olsson, C. Increased transcription levels induce higher mutation rates in a hypermutating cell line. J Immunol 166, 5051-7 (2001).
Bemark, M. & Neuberger, M. S. Oncogene 19, 3404-10 (2000).
Bowers, J. et al. Analysis of yeast MSH2-MSH6 suggests that the initiation of mismatch repair can be separated into discrete steps. J Mol Biol 15, 327-338 (2000).
Connor, A., Wiersma, E. & Shulman, M. J. On the linkage between RNA processing and RNA translatability. J Biol Chem 269, 25178-84 (1994).
Davidson, N. O. & Shelness, G. S. Annu Rev Nutr 20, 169-93 (2000).
Denépoux, S. et al. Induction of somatic mutation in a human B-cell line in vitro. Immunity 6, 3546 (1997).
Fischer, R. et al. Towards molecular fanning in the future: Pichia pastoris-based production of single-chain antibody fragments. Biotechnol Appl Biochem 30, 117-120 (1999).
Golding, G. B., Gearhart, P. J. & Glickman, B. W. Genetics 115, 169-76 (1987).
Gossen, M. & Bujard, H. Tight control of gene expression in mammalian cells by tetracycline-responsive promoters. Proc Nat'l Acad Sci USA 89, 5547-5551 (1992).
Gossen, M. et al. Transcriptional ativation by tetracyclines in mammalian cells. Science 268, 1766-1769 (1995).
Green, N. S., Verdugo, G., Getman, M. E., Scharff, M. D. Ig V Region Hypermutation in B-cell Hybrids Mimics In Vivo Mutation and allows for Isolation of Clonal Variants. Mol. Immunol 34, 1095-1103(1997).
Green, N. S., Lin, M. M., Scharff, M. D. Ig hypermutation in cultured cells. Immunol Review 162, 77-87 (1998).
Harris, R. S., Sale, J. E., Petersen-Mahrt, S. K. & Neuberger, M. S. Curr Biol 12, 435-8 (2002).
Kinoshita, K. & Honjo, T. Linking class-switch recombination with somatic hypermutation. Nat Rev Mol Cell Biol 2, 493-503 (2001).
Kobrin, B. J., Casadevall, A., Pirofski, L.-A., Schiff, C. & Scharff, M. D. in Somatic hypermutation in V regions (ed. Steele, E. J.) pp. 11-28 (CRC Press, Boca Raton, Fla., 1990).
Kohler, G. & Milstein, C. Continuous cultures of fused cells secreting antibody of predefined specificity. Nature 256, 495-7 (1975).
Kuppers, R. & Dalla-Favera, R. Oncogene 20, 5580-94 (2001).
Lin, M. M., Zhu, M. & Scharff, M. D. Sequence dependent hypermutation of the immunoglobulin heavy chain in cultured B-cells. Proc. Natl. Acad. Sci. USA 94, 5284-9 (1997).
Lin, M. M., Green, N. S., Zhang, W., Scharff, M. D. The effects of Eμ, 3′α (hs 1,2) and 3′κ enhancers on mutation of an Ig-VDJ-Cγ2a immunoglobulin heavy gene in cultured B-cells. International Immunology 10, 1121-1129 (1998).
Luria, S. E. & Delbruck, M. Mutation of bacteria from virus sensitivity to virus resistance. Genetics 28, 491-511 (1943).
Maizels, N. Cell 83, 9-12 (1995).
Martin, A. & Scharff, M. D. Nature Rev Immunol 2, 605-614 (2002a).
Martin, A. & Scharff, M. D. Somatic hypermutation of the AID transgene in B and non-B cells. Proc. Natl. Acad. Sci. USA 99, 12304-12308 (2002b).
Martin, A., Bardwell, P. D., Woo, C. J., Fan, M., Shulman, M. J. & Scharff, M. D. Nature 415, 802-6 (2002).
Michael, N., Martin, T. E., Nicolae, D., Kim, N., Padjen, K., Zhan, P., Nguyen, H., Pinkert, C. & Storb, U. Immunity 16, 123-34 (2002).
Muquan, L., Spira, G., and Scharff, M. D. Molecular Comparison of Cultured Hybridoma Cells that Switch Isotypes at High and Low Rates. Somatic Cell and Molecular Genetics 22, 329-340 (1996).
Muramatsu, M. et al. Specific expression of activation-induced cytidine deaminase (AID), a novel member of the RNA-editing deaminase family in germinal center B-cells. J Biol Chem 274, 18470-6 (1999).
Muramatsu, M., Kinoshita, K., Fagarasan, S., Yamada, S., Shinkai, Y. & Honjo, T. Cell 102, 553-63 (2000).
Muschen, M., Re, D., Jungnickel, B., Diehl, V., Rajewsky, K. & Kuppers, R. J Exp Med 192, 1833-40(2000).
Nelson, J. R. et al. Thymine-thymine dimer bypass by yeast DNA polymerase zeta. Science 272, 1646-1649 (1996).
No, D. et al. Proc. Natl Acad. Sci. USA 93, 3346-3351 (1996).
Okazaki Im, I., Kinoshita, K., Muramatsu, M., Yoshikawa, K. & Honjo, T. Nature (London) 416, 340-345 (2002).
Pasqualucci, L., Migliazza, A., Fracchiolla, N., William, C., Neri, A., Baldini, L., Chaganti, R. S. K., Klein, U., Kuppers, R., Rajewsky, K. & Dalla-Favera, R. Proc Natl Acad Sci USA 95, 11816-11821 (1998).
Pasqualucci, L., Neumeister, P., Goossens, T., Nanjangud, G., Chaganti, R. S., Kuppers, R. & Dalla-Favera, R. Nature 412, 341-6 (2001).
Peters, A. & Storb, U. Somatic hypermutation of immunoglobulin genes is linked to transcription initiation. Immunity 4, 57-65 (1996).
Petersen-Mahrt, S. K., Harris, R. S. & Neuberger, M. S. Nature 418, 99-104 (2002).
Phung, Q. H. et al. Increased Hypermutation at G and C Nucleotides in Immunoglobulin Variable Genes from Mice Deficient in the MSH2 Mismatch Repair Protein. J Exp Med 187, 1745-1751 (1998).
Poltoratsky, V., Goodman, M. F. & Scharff, M. D. J Exp Med 192, F27-F30 (2000).
Rada, C. & Milstein, C. The intrinsic hypermutability of antibody heavy and light chain genes decays exponentially. EMBO J 20, 4570-6 (2001).
Rada, C., Yelamos, J., Dean, W. & Milstein, C. The 5′ hypermutation boundary of kappa chains is independent of local and neighbouring sequences and related to the distance from the initiation of transcription. Eur J Immunol 27, 3115-20 (1997).
Rada, C., Ehrenstein, M. R., Neuberger, M. S. & Milstein, C. Hot Spot Focusing of Somatic Hypermutation in MSH2-Deficient Mice Suggests Two Stages of Mutational Targeting. Immunity 9, 135-141 (1998).
Reynaud, C. A. et al. Mismatch repair and immunoglobulin gene hypermutation: did we learn something? Immunol Today 20, 522-7 (1999).
Revy, P., Muto, T., Levy, Y., Geissmann, F., Plebani, A., Sanal, O., Catalan, N., Forveille, M., Dufoureq-Labelouse, R., Gennery, A., Tezcan, I., Ersoy, F., Kayserili, H., Ugazio, A. G., Brousse, N., Muramatsu, M., Notarangelo, L. D., Kinoshita, K., Honjo, T., Fischer, A. & Durandy, A. Cell 102, 565-75 (2000).
Rogozin, 1. B. & Kolchanov, N. A. Somatic hypermutagenesis in immunoglobulin genes. II. Influence of neighbouring base sequences on mutagenesis. Biochim. Biophy. Acta 1171, 11-8 (1992).
Rogozin, I. B., Pavlov, Y. I., Bebenek, K., Matsuda, T. & Kunkel, T. A. Somatic mutation hotspots correlate with DNA polymerase eta error spectrum. Nat Immunol 2, 530-6 (2001).
Rothenfluh, H. S. et al. Analysis of patterns of DNA sequence variation in flanking and coding regions of murine germ-line immunoglobulin heavy-chain variable genes: Evolutionary implications. Proc Nat'l Acad Sci USA 91, 12163-12167 (1994).
Sack, S. Z., Bardwell, P. D. & Scharff, M D. Testing the reverse transcriptase model of somatic mutation. Mol Immunol 38, 303-11 (2001).
Sale, J. E. & Neuberger, M. S. TdT-accessible breaks are scattered over the immunoglobulin V domain in a constitutively hypermutating B-cell line. Immunity 9, 859-69 (1998).
Shen, H. M. et al. Mutation of BCL-6 gene in normal B-cells by the process of somatic hypermutation of Ig genes. Science 280, 1750-1752 (1998).
Shen, H. M., Michael, N., Kim, N. & Storb, U. Int Immunol 12, 1085-93 (2000).
Spencer, J., Dunn, M. & Dunn-Walters, D. K. Characteristics of sequences around individual nucleotide substitutions in IgVH genes suggest different GC and AT mutators. J Immunol 162, 6596-601 (1999).
Spira, G., Gregor, P., and Scharff M. D. The use of chemiluminescence and the ELISA spot assay to identify and enumerate rare immunoglobulin switch variants. J. Immunol. Methods 165, 263-268 (1993).
Storb, U., Peters, A., Klotz, E., Kim, N., Shen, H. M., Hackett, J., Rogerson, B. & Martin, T. E. Immunol Rev 162, 153-60 (1998a).
Storb, U., Peters, A., Klotz, E., Kim, N., Shen, H. M., Kage, K., Rogerson, B. & Martin, T. E. Curr Top Microbiol Immunol 229, 11-9 (1998b).
Tao, W. & Bothwell, A. L. Development of B-cell lineages during a primary anti-hapten immune response. J Immunol 145, 3216-3222 (1990).
Tumas-Brundage, K. & Manser, T. The transcriptional promoter regulates hypermutation of the antibody heavy chain locus. J Exp Med 185, 239-250 (1997).
Vaswani, S. K. and Hamilton, R. G. Humanized antibodies as potential therapeutic drugs. Annals Allergy Asthma & Immunol. 81, 105-115.
Vora, K. A. et al. Severe attenuation of the B-cell immune response in Msh2-deficient mice. J Exp Med 189, 471481 (1999).
Wagner, S. D., Milstein, C. & Neuberger, M. S. Codon bias targets mutation. Nature 376, 732 (1995).
Washington, M. T. et al. Accuracy of lesion bypass by yeast and human DNA polymerase eta. Proc Nat'l Acad Sci USA 98, 8355-8360 (2001).
Wentworth, P. and Janda, K. D. Catalytic antibodies. Curr Opin Chem Biol 2, 138-144 (1998).
Wiesendanger, M., Kneitz, B., Edelmann, W. & Scharff, M. D. Somatic mutation in MSH3, MSH6, and MSH3/MSH6-deficient mice reveals a role for the MSH2-MSH6 heterodimer in modulating the base substitution pattern. J Exp Med 191, 579-584 (2000).
Wilkie, T. M. & Palmiter, R. D. Mol Cell Biol 7, 1646-55 (1987).
Worn, A. & Pluckthun, A. Stability engineering of antibody single-chain Fv fragments. J Mol Biol 305, 989-1010 (2001).
Wu, P & Claflin, L. Promoter-associated displacement of hypermutations. Int Immunol 10, 1131-1138 (1998).
Yelamos J. et al. Targeting of non-Ig sequences in place of the V segment by somatic hypermutation. Nature 20, 225-229 (1995).
Yoshikawa, K., Okazaki, I. M., Eto, T., Kinoshita, K., Muramatsu, M., Nagaoka, H. & Honjo, T. Science 296, 2033-6 (2002).
Zan, H. et al. Induction of Ig somatic hypermutation and class switching in a human monoclonal IgM+ IgD+ B-cell line in vitro: definition of the requirements and modalities of hypermutation. J Immunol 162, 3437-47 (1999).
Zan, H., Li, Z., Yamaji, K., Dramitinos, P., Cerutti, A. & Casali, P. J Immunol 165, 830-9 (2000).
Zan, H. et al. The translational polymerase zeta plays a major role in Ig and Bcl-6 somatic mutation. Immunity 14, 643-53 (2001).
Zeng, X. et al. DNA polymerase eta is an A-T mutator in somatic hypermutation of immunoglobulin variable genes. Nat Immunol 2, 537-41 (2001).
Zhang, W. et al. Clonal instability of V region hypermutation in the Ramos Burkitt's lymphoma cell line. Int Immunol 13, 1175-84 (2001).
Zhu, M., Rabinowitz, J. L., Green, N. S., Kobrin, B. J. & Scharff, M. D. Proc Natl Acad Sci USA 92, 28104 (1995).
Zuckier, L. S., Chang, C. J., Scharff, M. D., Morrison, S. L. Chimeric Human-Mouse IgG Antibodies with Shuffled Constant Region Exons Demonstrate that Multiple Domains Contribute to In-Vivo Half-Life. Cancer Research 58, 3905-3908 (1998).
Zuckier, L. S., Berkowitz, E. Z., Sattenberg, R. J., Zhao, Q. H., Deng, H. F., Scharff, M. D. Influence of affinity and antigen density on antibody localization in a modifiable tumor targeting model. Cancer Research 60, 7008-7013 (2000).
U.S. Pat. No. 6,121,424.
   Somatic Hypermutation and Isotype Switching

During the normal antibody response, B-cells initially express a highly diverse repertoire of IgM antibodies that have a low affinity for antigen and are unable to compete with cellular receptors and neutralize viruses or toxins. In order to make higher affinity antibodies, all vertebrates have evolved mechanisms to introduce mutations into the V regions of antibody genes that encode the antigen-binding site. This process is referred to as somatic hypermutation (SHM). The rates of V region mutation during this process are 10⁻⁵-10⁻⁴/base pair/generation, which is many orders of magnitude higher than the rates of mutation of other genes. Hot spot motifs that are preferentially targeted for mutation are concentrated in the complementarity determining regions (CDRs), or hypervariable regions, of the V region that encode the antigen binding site. Some of these base changes result in amino acid changes that increase the affinity and/or change the specificity of the antibody. In man and mouse, V region hypermutation occurs in centroblast B-cells in the germinal centers of secondary lymphoid organs. The B-cells with higher affinity compete effectively for antigen, present it to T cells and are stimulated to proliferate and differentiate and become the majority population in the germinal center. At the same time, in a process called class switching or isotype switching, these B-cells can rearrange the heavy chain V region gene from its initial location upstream from the μ constant region to the downstream γ, α, or ε constant regions. This allows the same antigen binding site to be expressed with each of those isotypes, to carry out the full panoply of antibody functions and to be distributed throughout the body. During the course of the primary antibody response, these mutational and isotype switching events begin to occur around 7 days after immunization and continue until about 10 days, after which these B-cells cease mutating and isotype switching and leave the germinal center to differentiate into plasma cells and memory B-cells.

The molecular and biochemical mechanisms responsible for V region hypermutation and isotype switching are poorly understood. However, in the last few years a few of the enzymes involved in both processes have been discovered. One of these is activation-induced cytidine deaminase, or AID, which is normally expressed exclusively in germinal center B-cells (Muramatsu et al., 1999). Mice genetically defective in AID and a subset of hyper-IgM patients with mutations in AID do not undergo SHM or isotype switching.

AID has 50% amino acid homology with APOBEC, which is an RNA editing cytidine deaminase that creates a stop codon in the mRNA for apolipoprotein B (apoB). This results in a truncated protein with a change in function that is required for the normal function of apoB. It is not known whether AID in B-cells acts directly on the DNA of the V region gene to produce mutations or as an RNA editing enzyme to activate proteins that are required for V region mutation and isotype switching (Kinoshita and Honjo, 2001).

Recent studies have shown that lowering the level of expression of AID by as little as 5-fold is associated with loss of V region mutation in cultured germinal center Ramos Burkitt's lymphoma B-cells (“Ramos cells”)(Zhang et al., 2001). However, there are no published studies that establish that overexpressing AID in those cultured B-cells that had lost the ability to mutate turns on mutation again at the wild type rate. There are also no published studies indicating that expressing AID in plasma cells or in non-B-cells turns on the SHM process.

Monoclonal Antibodies

Monoclonal antibodies have become valuable research and therapeutic tools. They are routinely generated by fusing antibody forming B-cells from animals or humans to continuously growing myeloma or other malignant B-cells. The resulting “hybridomas” produce monoclonal antibodies having homogeneous binding sites that are used as scientific and manufacturing reagents and in the diagnosis, prevention and treatment of disease. Monoclonal antibodies can also be generated by immortalizing B-cells with viruses (e.g., EBV) or by expression of oncogenes, or transfecting immunoglobulin genes into already established cell lines.

Six monoclonal antibodies have been approved by the FDA for therapeutic use and more than 60 others are in clinical trials. In addition, many other mouse and human monoclonal antibodies are currently being used as research and diagnostic reagents, including the production of macromolecules. New monoclonal antibodies are being routinely generated in numerous laboratories.

One of the persistent problems with the current state of hybridoma technology is that most monoclonal antibodies produced are of low affinity (i.e., they bind antigen relatively weakly). This is because relatively synchronized, early blasting B-cells are the cells that are most likely to form viable hybrids with the cultured myeloma cells. The B-cells that arise early in the primary or even the secondary response have not undergone as many rounds of somatic mutation, and more highly mutated B-cells are only rarely captured as hybridomas during the fusion process. In addition, some potentially useful monoclonal antibodies have cross-reactivities with self-antigens, or other cross-reactivities that make them less useful. The specificity to the antigen used to induce the antibody may also be undesirably high (i.e., does not bind to epitopes that are very similar, to which binding is desired) or undesirably low (i.e., binds excessively to similar epitopes).

Hybridoma cells that make monoclonal antibodies are plasma cells, which normally undergo very low rates of V region mutation. Mutants of hybridoma cells that arise in culture are almost all deletions in the constant region (Kobrin et al., 1990), and the few variable region mutants that have been identified arise at frequencies lower than 10⁻⁶ (Id.).

Previous studies have established that hybridomas can be switched in culture to express other isotypes. The frequency of such class switches can be increased by selecting for higher switching subclones (Muquan et al., 1996). Additionally, cultured hybridoma cells transfected with Ig genes can support rates of mutation that have been recorded at 10⁻⁴-10⁻⁵/bp/gen (Green et al., 1998). However, those transfected cells contained multiple copies of the Ig gene, and the recorded rate was for a single nonsense mutation that was embedded in a hot spot for mutation. We now estimate that the overall rate of mutation of the average base in the V region in those studies was 20-100 fold lower. Nevertheless, those studies do show that hybridoma cells can undergo rather high rates of V region mutation. In addition, if one fuses a non-mutating hybridoma (e.g., NSO—the fusion partner) to a mutating pre-B-cell (18-81), some hybrids have high rates of mutation (Green et al., 1997). Stable highly mutating clones can also be isolated (Id.). However, 18-81, which has recently been shown to express AID, is the only pre-B-cell that has ever been shown to mutate constitutively in culture. However, no published studies have suggested that AID is the sole factor required to induce high rates of mutations in the B-cells or hybrid cells, or that antibody genes in any hybridoma cell culture can be made to undergo high rates of mutation. The literature on mutation in plasma cells in culture is reviewed in Kobrin et al., 1990 and Green et al., 1998.

In many cases, if an existing monoclonal antibody could be altered to produce a higher affinity towards a specific antigen, it would be more effective and could be used in smaller amounts, thus reducing its cost. Higher affinity monoclonal antibodies would be especially useful to therapeutically target tumors (Zuckier et al., 2000) or neutralize viruses or toxins that bind to high affinity cellular receptors. Higher affinity monoclonal antibodies would also be useful in the prevention and treatment of infection with viruses such as Ebola and Lhasa Fever, or other agents that could be used as germ warfare agents. High affinity monoclonal antibodies could also be used against a variety of toxins such as botulinus and ricin for similar purposes.

In some cases, diagnostic or therapeutic monoclonal antibodies have cross-reactivity to a self antigen, which can produce toxicity or interfere with a diagnostic assay. If random mutation of the binding site were possible, variants without that cross reactivity could be identified and isolated.

The generation of monoclonal antibody class switching would also be useful. IgGs have a longer half-life than IgM and penetrate tissues and the placenta better. Human IgG1, 2, and 4 have half-lives of 25 days while IgG3 has a half life of only 7 days (Zuckier et al., 1998). Long or short half-lives have different benefits in different situations, making one or the other of these isotypes more useful. Also, IgA antibodies are particularly resistant to gut proteases. For all of these reasons, the ability to induce high rates of somatic mutation and isotype switching in vitro would make monoclonal antibodies more useful. The present invention satisfies that need by providing methods and compositions for inducing SHM in antibody genes in hybridomas as well as in other proteins in other cells.

### SUMMARY OF THE INVENTION

Accordingly, the inventors have discovered that the expression of activation-induced cytidine deaminase (AID) in eukaryotic cells causes increased rates of mutation of genes expressed in the eukaryotic cells, when the genes are operably linked to a promoter, and when the promoter is within about two kilobases of the gene. Based on that discovery, methods and reagents are provided to routinely create mutations in any gene that can be expressed in eukaryotic cells, including in particular antibody genes in monoclonal antibody-producing hybridomas. AID expression can also cause isotype class switching.

Thus, in some embodiments, the present invention is directed to methods of inducing a mutation in a gene in a eukaryotic cell, where the gene is operably linked to a promoter, and where the gene is within about two kilobases of the promoter. The methods comprise expressing a transgenic activation-induced cytidine deaminase (AID) gene in the cell.

In other embodiments, the invention is directed to methods of determining the effect of mutations in a gene encoding a protein on the phenotype of the protein in a eukaryotic cell. In these methods, the gene is operably linked to a promoter, and is within about two kilobases of the promoter. The methods comprise expressing the protein and a transgenic AID gene in the eukaryotic cell, establishing clonal colonies of the cell, identifying clonal colonies that produce a gene of the protein that has a mutation, determining whether the protein expressed by the mutated gene in any clonal colonies identified has an altered phenotype, and associating the altered phenotype with a particular mutation.

Additionally, the invention is directed to methods of inducing a mutation in an antibody gene in a eukaryotic cell. The methods comprise expressing a transgenic AID gene in the cell.

The present invention is also directed to methods of inducing a class switch in an antibody gene in a eukaryotic cell. The methods comprise expressing a transgenic AID gene in the cell.

In additional embodiments, the invention is directed to methods of altering an affinity or a specificity of a monoclonal antibody to an antigen, or altering a cross-reactivity of the monoclonal antibody to a second antigen. In these methods the monoclonal antibody is produced by a eukaryotic cell that is capable of expressing a transgenic AID gene under inducible control. The methods comprise expressing the AID gene in the eukaryotic cell for a time and under conditions sufficient to induce a mutation in a gene encoding the monoclonal antibody, suppressing expression of the AID gene in the eukaryotic cell, establishing clonal colonies of the cell, and determining whether the monoclonal antibody produced by any of the clonal colonies of the cell has altered affinity or specificity to the antigen, or altered cross-reactivity to the second antigen.

The present invention is additionally directed to various eukaryotic cells that comprise an AID gene. In some of these embodiments, the AID gene is transgenic. In those eukaryotic cells, expression of the AID gene is preferably inducible. Cells envisioned in these embodiments include myeloma fusion partners and hybridomas that express an AID gene. In other embodiments the eukaryotic cells expressing the AID gene are not B-cells.

The invention also encompasses the mutated genes produced by the above-described methods and cells, the proteins encoded by those mutated genes, and cells that comprise those genes or proteins.

### BRIEF DESCRIPTION OF THE DRAWINGS

FIG. 1 summarizes experimental results which establish that human AID (HAID) expression induces SHM in non-mutating Ramos cells. RNA was isolated from (a) selected subclones of Ramos cells (Zhang et al., 2001) and (b) stable transfectants of the non-mutating Ramos clone 1 that overexpress hAID. HAID and GAPDH were reverse transcribed, and five fold dilutions of the resulting cDNA were amplified by PCR. Levels of AID mRNA in clones 6 and 7 are about 5-fold higher than in clone 1 (Zhang et al., 2001) (Panel a), and are ˜25 fold higher in clones A.2 and A.5 than in C.1 and A.1 (Panel b). The mutation rates shown for the subclones in Panel a are taken from Zhang et al., 2000, and in Panel b are from the present work.

FIG. 2 summarizes experimental results which show the induction of SMH in hybridomas transfected with AID. Panel a. Hybridomas N89 (nonsense in leader) and N114 (nonsense in V-region) were transfected with empty vector or the hAID construct. Frequency of nonsense revertants, as detected with the ELISA spot assay (Spira et al., 1993), is plotted. Typical ELISA spots for N89 are shown in inset. The difference in the frequencies of reversion of N114 vector and N114 hAID was statistically significant (P<0.05). Panel b. Northern blots for hAID and GAPDH of N89, N114, and P1-5 transfected hybridoma clones. Clone numbers for N89 and N114 clones correspond to numbers in Panel a.

FIG. 3 summarizes mutation data observed in hybridoma clones. Panel a. Pie charts, as previously shown (Sale and Neuberger, 1998), depicting the distribution of the frequencies of mutation of P1-5 and N114 hybridoma clones. Shown are the number of sequences analyzed (center of pie) and the proportion of sequences with 0, 1, 2 . . . mutations (pie slices). Panel b. All mutations located within the V-region (V186.2) of hybridoma P1-5 clones are shown. Duplicate mutations were counted once in Table 1, unless genealogies indicate mutations were unique. Hotspot motifs (RGYW and WRCY) are bolded. Although other hot spot motifs are frequently mutated, we and others have observed a high frequency of mutation at the codon 31 hotspot (underlined) in vivo (Sack et al., 2001).

FIG. 4 summarizes results of experiments establishing that AID induces mutations in cells other than B-cells, here T cells (Bw-5147) and Chinese hamster ovary cells (CHO). CHO and Bw5147 cells were stably-transfected with the same immunoglobulin heavy and light chain genes used previously (Lin et al., 1998). The heavy chain gene (Igγ2a) has a nonsense codon within the V-region, and thus cells cannot produce functional IgG2a unless the nonsense codon is mutated. These cells were transfected with empty vector (open circles) and vector expressing hAID (filled in circles). The ELISA-spot assay was used to assay for cells secreting IgG2a that have reverted the nonsense codon of the heavy chain gene. Because some clones transfected with hAID failed to express AID (see lower panel: Northern blots for AID and GAPDH), the data points from such clones were placed with the empty vector-transfected clones. Thus, the columns are divided in clones that are AID-negative and clones that are AID-positive. Analysis of the primary data by the independent samples t-test (with equal variances assumed) shows that the reversion frequencies between the AID−ve and AID+ve are statistically significant (p<0.05 and p<0.01 for Bw5147 and CHO, respectively).

FIG. 5 summarizes results from experiments establishing that AID hypersensitizes Ramos cells to class-switch recombination. Indicated Ramos clones were incubated with empty-vector-transfected NIH3T3 cells (− stimulation) or with CD40L-transfected NIH3T3 cells and 5 ng/ml of IL-4 (+ stimulation) for 10 days. Panel A shows results of ELISA testing of supernatants for secreted IgG and IgM. Panel B shows results of RT-PCR analysis for IgG mRNA and the sterile transcripts Iγ1, 2, 3 on 10-day stimulated and unstimulated clones.

FIG. 6 summarizes results from experiments showing mutations in the AID transgene from Ramos, hybridoma P1-5, and CHO cells. Mutations located within the AID transgene from the Burkitt's lymphoma Ramos (upper case), hybridoma P1-5 (lower case), and CHO cells (upper case bolded). Duplicate mutations from each clone were counted once in Table 3. Hotspot motifs (RGYW and WRCY) are bolded.

FIG. 7 summarizes experimental results establishing that AID induces SHM in CHO cells. Panel A. Murine Vn/ECMV γ2a-construct transfected into CHO cells to study SHM. Previously described in Lin et al., 1998, this heavy chain immunoglobulin construct has replaced the intronic μ enhancer with a CMV enhancer, and contains a TAG nonsense codon within an RGYW hot-spot motif at codon 38. Panel B. Left two columns: CHO clone CHO-LC18 (see Materials and Methods) stably transfected with heavy and light chain immunoglobulin genes was transfected with empty vector (open circles) or the hAID construct (filled in circles). Depending on expression of AID (see below), data was distributed into AID-negative (AID−) and AID-positive (AID+) columns. Frequency of nonsense revertants, as detected with the ELISA spot assay, is plotted. Right two columns: 10 subclones of CHO clones A.3 and A.9 were further analyzed using the ELISA spot assay to calculate mutation rates by fluctuation analysis.

### DETAILED DESCRIPTION OF THE INVENTION

The present invention is based on the discovery that the expression of activation-induced cytidine deaminase (AID) in eukaryotic cells causes increased rates of mutation of genes expressed in the cells, when the genes are operably linked to a promoter, and when the promoter is within about two kilobases of the gene. This discovery makes possible the development and use of methods and reagents to routinely create mutations in any gene that can be expressed in eukaryotic cells, including native genes and genes introduced into the cell transgenically. In particularly useful embodiments, expression of AID in monoclonal antibody (Mab)-producing hybridomas causes high rates of mutation and class switching in the antibody genes, allowing the selection of monoclonal antibodies with altered affinity, specificity, cross-reactivity to an antigen, or isotype.

It has also been discovered that the mutating effects of AID on a gene can be negated by flanking the gene, either at the 5′ or the 3′ end, with foreign sequences (i.e., sequences not native to the cell). See, e.g., Example 5. In these embodiments, the flanking sequence is at least about 200 bp, more preferably at least about 1000 bp, and most preferably at least about 2000 bp. In the most preferred embodiments, the foreign sequence flanks both the 5′ and the 3′ end of the gene. It is also preferred that the foreign sequences are sequences are from a species that is highly unrelated to the cell, e.g., yeast sequences when the cell is a mammalian cell. In the most preferred embodiments, the sequences are bacterial (e.g., E. coli) sequences.

This discovery is particularly useful for creating cells or organisms comprising a transgenic AID gene that is not subject to mutation by its own gene product. Thus, by retaining the parts of a plasmid vector which has foreign (e.g., bacterial) DNA sequences flanking the AID gene, and stably integrating those foreign sequences with the AID gene into the host cell, AID can be introduced and overexpressed in a way that prevents the AID gene product from mutating the introduced AID transgene.

Activation-induced cytidine deaminase (“AID”) genes useful for the methods and compositions of this invention can be any vertebrate AID gene, defined herein as a cytidine deaminase that is naturally induced upon activation of B-cells in the vertebrate. In preferred embodiments, the AID gene is a mammalian AID gene, as exemplified in GenBank accession numbers NM020661 (human) or NM009645 (mouse). In the most preferred embodiments, the AID gene is a human AID gene.

As used herein, “mutation” refers to an alteration in a basepair of a gene (also known as a point mutation, e.g., C to T) or the alteration in the amino acid sequence of a protein as a result of the alteration in the gene sequence. The gene mutation can cause the generation of a premature stop codon in the gene, causing a truncated protein, or no protein, to be synthesized.

As used herein, the terms “gene expression” and “protein expression” are synonymous and refer to the transcription and translation of a gene into a protein encoded by the gene.

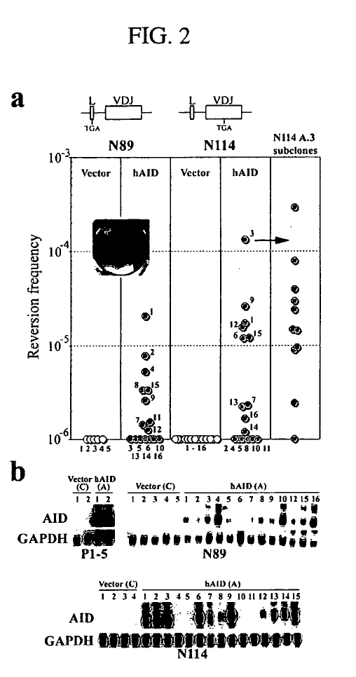

According to the present invention, expression of AID in a eukaryotic cell causes mutations in the eukaryotic cell. This discovery allows the development of methods of inducing a mutation in a gene in a eukaryotic cell. The methods comprise expressing a transgenic activation-induced cytidine deaminase (AID) gene in the cell. In these methods, the gene that is subject to mutation is any gene that is operably linked to a promoter, where the gene is within about two kilobases (kb) of the promoter. See Rothenfluh et al., 1994; Rada and Milstein, 2001. In preferred embodiments, the gene is also operably linked to an enhancer, since enhancers increase expression of the gene to the high levels needed to achieve measurable mutation by the expressed transgenic AID. The gene is also preferably between 10 bases and 2 kb in the 3′ direction from the promoter. See Wu and Claflin, 1998. See also Example 1 and FIG. 2, where some of the mutations generated targeted very near the start of transcription.

As used herein, a transgenic gene or a transgene is a gene that is present in a cell due to molecular genetic manipulation. The gene can be integrated into the genome or the cell or present in the cell extrachromosomally, e.g., as part of a plasmid or virus. The gene can be stably maintained in the cell or transiently maintained, then lost from the cell.

In the methods of the present invention, the mutation of the gene caused by AID expression in the cell is an average mutation rate in the gene that is at least twice that of the mutation rate of that gene without AID. In preferred embodiments, the mutation rate is at least 5 times, more preferably 10 times, still more preferably 50 times the average mutation rate of the gene in the cell not expressing AID. In the most preferred embodiments, the average mutation rate is at least 100 times the average mutation rate of the gene in the cell not expressing AID.

In the methods of this invention, AID-induced mutation occurs more frequently where the nucleic acid sequence of the gene corresponds to the well known “hot spot” sequences of variable regions of antibody genes. Those hot spots have the sequences are RGYW or WRCY (R=A or G, Y=C or T, W=T or A). Thus, genes that have higher incidences of these hot spot sequences would be expected to have higher mutation rates than those genes that have fewer hot spot motifs. Thus, mutation rates at any particular basepair, particularly at the G of an RGYW motif, or a C of a WRCY motif, can be 1000 times, or more, the mutation rate at that basepair in the absence of AID.

Any promoter or enhancer known in the art that allows the expression of the gene in the eukaryotic cell can be used for these methods. Preferably, the promoter allows moderate to high expression of the gene in the cell. The amount of expression can be measured by any means known in the art, including quantitative measurement of the gene product, or preferably quantitation of polyA mRNA. A useful measurement of gene expression of a particular gene is the determination of the relative amount of polyA mRNA of the gene compared to total mRNA in the cell. The skilled artisan can make this determination without undue experimentation using well-known methods. In preferred embodiments, the gene to be mutated comprises at least 0.01% of total polyA mRNA in the cell. In more preferred embodiments, the polyA mRNA of the gene comprises at least 0.1% of total polyA mRNA in the cell. In still more preferred embodiments, the polyA mRNA of the gene comprises at least 0.5% of total polyA mRNA in the cell. In the most preferred embodiments, the polyA mRNA of the gene comprises at least 1% of total polyA mRNA in the cell. For B-cells, a preferred promoter is an immunoglobulin promoter and, where present, a preferred enhancer is an immunoglobulin enhancer. For other mammalian cells, preferred promoters and enhancers (where present) are viral promoters and enhancers, many suitable examples of which are known in the art.

In these methods, the AID gene can be expressed constitutively in the cell. In preferred embodiments, however, the AID gene expression is inducible. Inducible AID expression is preferred because this allows the generation of mutants when AID is expressed, and then the selection and evaluation of the generated mutants when AID is not expressed, thus avoiding the possibility of the further generation of mutants during the selection and evaluation steps, or at other times when mutation is not wanted.

In embodiments where the AID gene is inducible, the system used to control induction is not narrowly limited, and can be selected by the skilled artisan without undue experimentation based on particular characteristics of the cell type and gene in which mutation is desired. Among the preferred induction systems for mammalian cells is the well-known positive or negative regulatory tet system (Gossen and Bujard, 1992; Gossen et al., 1995) and the ecdysone receptor-inducible system (See, e.g., No et al., 1996; Albanese et al., 2000).

Because the accessory proteins and enzymes associated with SHM, i.e., MSH2, MSH6, and polymerases ζ and η are present in all eukaryotic cells (see, e.g., Bowers et al., 2000; Nelson et al., 1996; Washington et al., 2001, describing these proteins and enzymes in yeast), it is expected that any eukaryotic cell can be utilized in these methods. Included are yeast and plant cells. In preferred embodiments, the eukaryotic cell is a vertebrate cell. In more preferred embodiments, the cell is a mammalian cell, including mouse, rat and human cells. Any eukaryotic cell type that can be cultured is expected to be useful for these methods. See also Example 2, where gene mutation is induced in Chinese hamster overy (CHO) cells and T cells expressing an AID transgene. In some preferred embodiments the cell can be a B-cell, for example a hybridoma expressing an antibody gene to which mutation is desired.

In these methods, the gene subject to mutation can be a native gene (i.e., a gene in its natural chromosomal or extranuclear location in the cell), provided that the gene is operably linked to a promoter and, preferably, an enhancer, and the gene is within about 2 kb of the promoter.

Alternatively, the gene subject to mutation can be a transgene introduced into the cell transiently or stably, and integrated into the genome or present in an extranuclear vehicle such as a plasmid or a virus. The gene can also be of prokaryotic or eukaryotic origin, e.g., from a microbe, plant, insect, vertebrate, etc., including a mammal or a human. It is well established that any gene can be mutated by SHM mechanisms if properly positioned and operably linked to a promoter and, preferably, an enhancer. See, e.g., Peters and Storb, 1996; Yelamos et al., 1995; Tumas-Brundage and Manser, 1997; and Shen et al., 1998. Martin and Scharff (2002), provided herein as Example 4, further confirms the expectation that non-immunoglobulin genes, including the AID transgene itself, are subject to AID-induced SHM, both in B cells and non-B cells.

These methods of causing a mutation in a gene can be used, for example, to determine the effect of the generated mutations in the structure or function of the protein encoded by the gene. The methods can also be used to create mutants of a protein that has desirable altered characteristics. Nonlimiting examples include mutants of binding proteins such as antibodies, cytokines or transcription factors, where the mutants have altered specificity or affinity or block the effect of the binding protein; mutants of enzymes, where the mutants have altered catalytic activities or environmental optima; mutants of toxins, where the mutants have altered toxicity or antitoxin activity; and mutants of structural proteins, where the mutants affect cellular or tissue morphology.

Thus, the present invention is also directed to methods of determining the effect of mutations in a gene encoding a protein on the phenotype of the protein in a eukaryotic cell. As in the previously described methods, the gene to be mutated must be operably linked to a promoter and an enhancer, and within about two kilobases of the promoter. The methods comprise the following steps:
(a) expressing the protein and a transgenic AID gene in the eukaryotic cell;
(b) establishing clonal colonies of the cell;
(c) identifying clonal colonies that produce a gene of the protein that has a mutation;
(d) determining whether the protein expressed by the mutated gene in any clonal colonies identified in step (c) has an altered phenotype; and
(e) associating the altered phenotype with a particular mutation.

The above steps need not be performed in the order set forth.

As with the previously described methods, these methods can employ any eukaryotic cell that can be cultured, and any gene from any source. Any promoter and enhancer (when employed) can also be used, but preferred are those that allow moderate to high expression of the gene. Inducible AID expression is preferred, e.g., using a let or ecdysone receptor system, so that AID expression can be induced only during step (a) to avoid generation of mutants during the subsequent steps.

For these methods or any other methods described herein, the identification of mutants as in step (c) can be by any means suitable for the gene and cell type involved. In some embodiments, the entire gene from each clonal colony can be sequenced, e.g., after PCR amplification. Alternatively, only a portion of the gene can be sequenced, for example the portion encoding the active site of an enzyme or a binding protein.

In other embodiments of these methods or other invention methods, the colonies harboring mutant genes can be identified by changes in the expressed mutant protein encoded by the gene, or, where appropriate, changes in cell or colony phenotype engendered by the mutation. Those clones expressing the desired phenotype are then sequenced to determine the mutation that is causing the phenotypic change. In the present methods, this is equivalent to performing step (d) before step (c). The clonal colonies can be screened, for example, for visible changes in the colony or cell morphology, or for changes in binding of antibodies to the protein, such as an elimination, reduction, increase, or commencement of antibody binding. A useful assay for screening with antibodies is the ELISA spot assay (Spira et al. 1993).

For these methods or any other methods described herein, once the target gene is induced to undergo reasonably high rates of mutation (and/or isotype switching—see below) in tissue culture, there are many techniques that allow even relatively rare subclones expressing a desired protein to be separated from the rest of the cells and then propagated to produce the mutant protein. When the protein is an antibody, useful methods include enrichment of cells producing high affinity antibodies by FACS after staining with limiting amounts of fluorescent-labeled antigen or tetramers, use of antigen coated beads to enrich for higher affinity subclones and sib selection using the ELISA spot assay to screen for variants. Similar techniques using anti-isotype specific antibodies are routinely used to isolate isotype switch variants (Spira et al., 1993).

The above methods are particularly useful for inducing mutations in antibody genes. Thus, some embodiments of the invention are directed to methods of inducing a mutation in an antibody gene in a eukaryotic cell. The methods comprise expressing a transgenic AID gene in the cell. As with previous methods, the antibody gene can be native to the cell, for example as in a hybridoma cell. Alternatively, the antibody gene can be a transgene in any eukaryotic cell, such as mammalian cells (e.g., CHO or T cells—see Example 2), yeast cells, plant cells, insect cells, vertebrate cells, etc. The antibody gene can be from any vertebrate species, for example, rat, mouse, rabbit, hamster, or human.

The antibody gene can be genetically unaltered before the AID gene is expressed, i.e., a heavy or light chain gene as are naturally made in B-cells. Alternatively, the antibody gene can be altered by any means known in the art, for example as with humanized antibodies (Vaswani and Hamilton, 1998), single chain antibodies and fragments (Fischer et al., 1999; Worn and Pluckthun, 2001) and multivalent antibodies (see, e.g. U.S. Pat. No. 6,121,424).

As in previous embodiments, these methods can utilize constitutive control of AID gene expression in the cells. However, inducible control, e.g., using a let or ecdysone receptor system, is preferred.

For most practical purposes, it is preferred that the antibody gene in these embodiments encode at least a portion (e.g., a light chain or a heavy chain) of an antibody that binds to an antigen. Both light chain and heavy chain antibody genes can also be mutated.

The methods of the invention are not limited to the mutation of antibody genes encoding antibodies that bind to any particular antigen. For example, the mutated antibody gene can encode at least a portion of a catalytic antibody (i.e., an antibody that catalyzes a chemical reaction [Wentworth and Janda, 1998]). The mutated antibody gene can also encode at least a portion of an antibody that binds to a pathogen, for example an animal pathogen, e.g., a human pathogen. The pathogen can be a bacterium, virus, or any other organism. The antigen can also be a toxin, such as polypeptide toxins produced by microorganisms or plants (e.g., ricin). The antigen can also usefully be an enzyme, a transcription factor, a cytokine, a structural protein, or any other protein. Antibodies to any macromolecules such as carbohydrates, nucleic acids, lipids, and small chemicals such as haptens are also envisioned as benefitting from these methods.

The mutant antibody produced as a result of these methods can have any of a number of alterations in its antigen binding capacity. It can have higher or lower affinity for the antigen than before the mutation. It can also have higher or lower specificity for the antigen than before the mutation. Additionally, it can have altered cross-reactivity (either increased or decreased) for a second antigen than before the mutation.

As is well known, AID is required for both antibody class switching and somatic hypermutation in activated B-cells. However, there are no published studies showing that expression of AID in plasma cells or in B-cells at other stages of differentiation induces class-switch recombination. This is established in Example 3, which shows spontaneous class-switching caused by expression of AID. Therefore, the provision of AID in a eukaryotic cell expressing antibody heavy chain genes induces class switching if the genes of alternate classes are also present in the same configuration as those genes are present in a B-cell. The invention is thus directed to methods of inducing a class switch in an antibody heavy chain gene in a eukaryotic cell, the method comprising expressing a transgenic AID gene in the cell.

As with previous methods, it is preferred that the AID gene is under inducible control (e.g., with a tet system), although constitutive control is also envisioned. Also as with previous methods, any eukaryotic cell can be utilized in these methods, provided the cell harbors both the antibody gene and at least one gene for the isotype to which the switch is desired, in the B-cell configuration required for class switching. In preferred embodiments, the cell is a myeloma cell, most preferably a hybridoma cell, since those cells already make antibody genes and comprise properly configured alternative classes.

As with previous methods, antibodies from any species, as well as genetically altered (e.g., humanized) antibodies can be employed in these methods. Also, the methods are not narrowly limited to antibodies having any particular antigen specificity, and includes catalytic antibodies, antibodies to pathogens or toxins, or antibodies to haptens, enzymes, transcription factors, cytokines, and structural proteins.

In related embodiments, the present invention is directed to methods of altering an affinity or a specificity of a monoclonal antibody to an antigen, or altering a cross-reactivity of the monoclonal antibody to a second antigen. These methods require the monoclonal antibody to be produced by a eukaryotic cell that is capable of expressing a transgenic AID gene under inducible control. The methods comprise
(a) expressing the AID gene in the eukaryotic cell for a time and under conditions sufficient to induce a mutation in a gene encoding the monoclonal antibody;
(b) suppressing expression of the AID gene in the eukaryotic cell;
(c) establishing clonal colonies of the cell; and
(d) determining whether the monoclonal antibody produced by any of the clonal colonies of the cell has altered affinity or specificity to the antigen, or altered cross-reactivity to the second antigen.

The preferred cells for these methods are hybridoma cells, although any eukaryotic cell could be usefully employed. As with previous methods, these methods are not limited to use with antibodies from any particular species, or binding any particular antigen.

In some embodiments of these methods, steps (a) through (d) are repeated with a clonal colony that has altered affinity or specificity to the antigen, or altered cross-reactivity to the second antigen. This allows the generation of clones that produce antibodies with several accumulated mutations.

The step (d) selection for particular clones of interest can be developed for any particular antibody by the skilled artisan without undue experimentation. For example, where an antibody that has greater or less specificity is desired, the candidate clones can be screened with a labeled antigen and antigens of similar structure, either separately, or in competition with each other. Myriad other assays can be easily developed to select antibodies with increased or decreased affinity to the antigen, or increased or decreased cross-reactivity with a second antigen.

In other embodiments, the invention is directed to eukaryotic cells comprising a transgenic AID gene, wherein expression of the AID gene is inducible. These embodiments are not narrowly limited to any particular induction system, and any appropriate system can be adopted by a skilled artisan without undue experimentation. For mammalian cells, a preferred system is the tet system (either inducible or repressible by doxycycline or analogs) or an ecdysone receptor system, since these systems afford very tight regulatory control.

The cells of these embodiments can be any eukaryotic cell, including but not limited to yeast, insect, vertebrate or mammalian (including human) cells. Among preferred mammalian cells are Chinese hamster ovary (CHO) cells, T cells, or myeloma (including hybridoma) cells.

Since the cells of these embodiments are able to cause mutations in expressed genes, the cells can further comprise a gene encoding a protein, wherein the gene is operably linked to a promoter and, preferably an enhancer, and wherein the gene is within about two kilobases of the promoter. In those cells the gene can be a native gene or a transgene. Preferably, the gene undergoes mutation upon expression of the AID gene. In some preferred embodiments of these cells, the gene is an antibody gene.

The invention is also directed to eukaryotic cells expressing an AID gene, wherein the cell is not a B-cell. In these cells, the AID gene can be a native gene, for example when the cells are created by cell fusion between a B-cell and a non-B-cell, and the cell derives its expression of AID from the B-cell. Thus, the cells of these embodiments can be hybrid cells that are partially B-cells. Examples of B-cells that can be used for these hybridizations are Ramos cells that express AID. See, e.g., Example 1.

In preferred embodiments of these cells, the AID gene is a transgene. As in previously described methods and cells, the AID can be constitutively expressed, or inducible, for example using a tet or ecdysone system.

The cells of these embodiments can be any eukaryotic cell as appropriate for any particular application. Nonlimiting examples include yeast cells, insect cells, and vertebrate cells, including mammalian (e.g., human) cells. They can also be any type of cell that can be maintained in culture. Preferred examples include T cells and CHO cells.

As with previously described embodiments, the cells of these embodiments can also comprise a gene, which can be a native gene or a transgene, operably linked to a promoter and an enhancer, wherein the gene is within about two kilobases of the promoter. Preferably, the gene undergoes mutation upon expression of the AID gene. A particularly useful example of the gene is an antibody gene.

Related to the above described cells, the invention is also directed to a myeloma fusion partner expressing an AID gene. As used herein, a myeloma fusion partner is a myeloma cell that can be grown in culture and that has a selection system that allows for the efficient selection of hybrid cells when the fusion partner is fused with a B-cell during the production of hybridomas. A preferred example of a selection system is a deficiency in HGPRT, which allows selection of the hybridoma on hypoxanthine-aminopterin-thymidine (HAT) media. Examples of commonly used myeloma fusion partners are Sp2/0-Ag 14, FOX-NY, P3X63, NX-1, P3, P3X643 Ag8.653, NS1, and NSO.

The myeloma fusion partners of these embodiments are useful in the production of hybridomas producing monoclonal antibodies that can be mutated when the AID gene is expressed. To produce such hybridomas, the practitioner need only fuse these fusion partners with B-cells using the usual hybridoma production protocol. Thus, these myeloma fusion partners allow the mutation of monoclonal antibodies in any hybridoma, without having to transfect the hybridoma with an AID gene.

The AID gene in the myeloma fusion partner can be native, which can be created by fusing an AID-producing Ramos B-cell with a myeloma fusion partner that is not producing AID. Preferably, however, the AID gene is a transgene.

The AID gene can be constitutively expressed. However, it is preferred that the AID gene is inducible, e.g., with tet or ecdysone selection, since those systems allow precise control of when mutations can be created in hybridomas produced using the cells.

The present invention is also directed to a hybridoma expressing an AID gene. Such a hybridoma can be created, for example, by fusing a non-AID expressing hybridoma with a cell expressing AID, such as a Ramos B-cell or a myeloma. Alternatively, a hybridoma that does not produce AID can be selected to produce AID. In preferred embodiments, the hybridoma is created by transfecting a hybridoma that does not express AID with a vector encoding an AID gene. Such vectors can be designed and created by the skilled artisan without undue experimentation. In other preferred embodiments, the hybridoma expressing AID is created by fusing a B-cell with the myeloma fusion partner previously discussed that is capable of expressing a transgenic AID gene. Thus, although the AID gene in these hybridomas can be native, it is preferred that it is transgenic. It is also preferred that AID gene expression be inducible in these hybridomas, although constitutive expression is also envisioned.

In preferred embodiments to these hybridoma cells, the hybridoma expresses an antibody that binds to an antigen. These embodiments are not limited to hybridoma cells expressing antibodies to any particular antigen, nor from any particular species.

In some embodiments of these hybridoma cells, the antibody gene expressed therein has undergone mutation upon expression of the AID gene to cause a mutation in the antibody. The mutation can cause a change in the antibody affinity or specificity to an antigen, or the cross-reactivity of the antibody to a second antigen. Alternatively, the mutation can cause no discernable change in the antibody binding characteristics. This lack of discernable change can be due to the mutation altering an amino acid residue that does not affect the antibody binding characteristics. The lack of change can also be due to the mutation being silent by changing a nucleotide residue that has no effect in the amino acid sequence due to the redundancy of the genetic code.

The antibody produced by the hybridomas in these embodiments can also have undergone a class switch during AID gene expression, with or without mutation in the antibody.

Other embodiments of the present invention includes mutated genes, including antibody genes, produced by any of the above methods; mutated proteins (including antibodies) encoded by any of those mutated genes; mutated genes and proteins produced by any of the above described cells; vectors useful for producing any of the above-identified cells; and eukaryotic cells comprising any of the mutated genes produced by the above methods or cells.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual” (1989); “Current Protocols in Molecular Biology” Volumes I-IV (Ausubel, R. M., ed. (1997); “Cell Biology: A Laboratory Handbook” Volumes I-III (J. E. Celis, ed. (1994); and “Current Protocols in Immunology” Volumes I-III (Coligan, J. E., ed. (1994).

### EXAMPLE 1

### Expression of AID and Somatic Hypermutation of Antibody Genes

In this Example, we establish that AID is required for somatic hypermutation (SHM) in centroblast-like Ramos cells. We then show that expression of AID is sufficient to induce SHM in hybridoma cells, which represent a later stage of B-cell differentiation that does not normally undergo SHM. Methods.

Cell lines, cell culture and transfection conditions: Ramos cells were grown as previously described (Zhang et al., 2001). N89 and N114 hybridoma cells were described previously (Connor et al., 1994), while the hybridoma P1-5 was obtained from Dr. Alfred Bothwell (Tao and Bothwell, 1990). Ramos cells were electroporated with 10 μg linearized DNA in IMDM medium at 250 volts, 960 μF, plated into 96 well plates, and selected with 0.6 mg/ml hygromycin B. The hybridoma cells were electroporated as described previously (Lin et al., 1997), and selected in 0.3 mg/ml hygromycin B. The ELISA spot assay was performed as previously reported (Zhang et al., 2001). Briefly, each drug resistant colony was expanded to ˜1-5×10⁶ cells, and plated onto 96 well plates that were pre-coated with anti-mouse IgM antibody. After 22 hours, the plates were developed for secreted IgM.

Constructs: Full length human AID (hAID) was amplified using primers previously reported (Zhang et al., 2001) and cloned into Zero BluntTOPO PCR Cloning Kit (Invitrogen) to sequence. The hAID insert was excised with EcoR1, blunted with Klenow polymerase, and cloned into pCEP4 (Invitrogen) digested with PvuII. Vectors were digested with Nru1 and EcoRV prior to transfection.

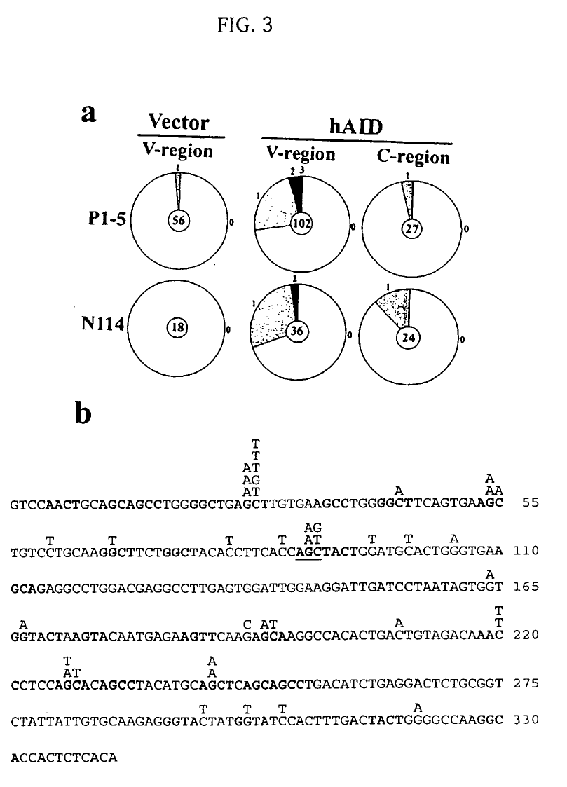

PCR Amplification, cloning, and sequencing V- and C-regions regions from cell lines: Genomic DNA was prepared as previously reported (Zhang et al., 2001). V-regions from the various B-cell lines were amplified with Pfu polymerase (Stratagene) from genomic DNA using 30 cycles of 95° C./15 sec, 56° C./15 sec, 72° C./30 sec. Primers for N114 V-region, 5′ primer: TTACCTGGGTCTATGGCAGT, 3′ primer: TGAAGGCTCAGAATCCCCC, and Cm2-3 region 5′ primer: CCCCTCCTTTGCCGACATCTTCC, 3′ primer: TTCCATTCCTC-CTCGTCACAGTC. Primers for Ramos and P1-5 V-region were published before (Zhang et al., 2001, Sack et al., 2001). Primers for P1-5 Cg1 exons 2-3: 5′ primer: CACACAGCTCAGACGCAACCCC, 3′ primer: GGATCATTTACCAGGAGAGTGGGAGAGG. This primer pair amplifies both the Balb/c and the C57Bl/6 Cg1 segments from the P1-5 hybridoma, which can be distinguished by allotypic differences. Since the NP-specific V-region of P1-5 derives from C57Bl/6 (Tao and Bothwell, 1990), only C-regions sequences from C57Bl/6 are reported in FIG. 3a. PCR products were cloned and sequenced as previously reported (Zhang et al., 2001).

Extraction of RNA, RT-PCR, and Northern Blots: ˜5×10⁶ cells were lysed with 1 ml Trizol reagent (GibcoBRL) and RNA extracted according to manufacturers instructions. ˜1 μg of total RNA was either run on formaldehyde gels for northern blots, or reverse transcribed using the Superscript II kit (GibcoBRL). 5 μl of the RT product was diluted 5-fold with H₂O sequentially 3 times. 1 μl of each of these 3 dilutions was used in a PCR reaction, and all amplifications of each cDNA from each different clone were done together. Taq polymerase (Roche) was used to amplify GAPDH and AID using primers and conditions previously described (Zhang et al., 2001).

Statistics: Statistics for sequencing data in Table 2 and primary data for reversion frequencies in FIG. 2a were measured by the independent-samples t-test with equal variances assumed (SPSS v.10).

Results

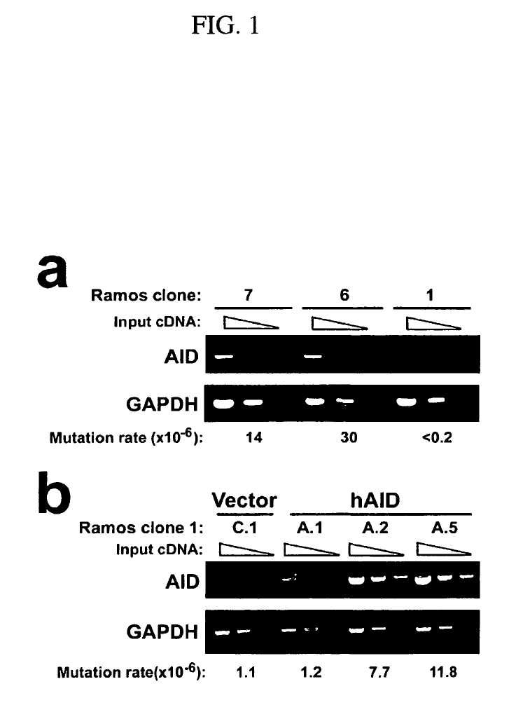

Three human B-cell lines (i.e. Ramos, BL-2 and CL-01) were recently shown to undergo SHM (Sale and Neuberger, 1998; Denépoux et al., 1997; Zan et al., 1999), thus opening the possibility of studing this process in vitro. In previous work (Zhang et al., 2001), we found that V-region mutation rates in different Ramos clones correlated with the level of their AID mRNA, suggesting that AID plays an important role in SHM in Ramos cells. Specifically, both the rates of mutation and the mRNA levels of AID for Ramos clones 6 and 7 were higher than those for Ramos clone 1 ((Zhang et al., 2001; FIG. 1a). To determine whether low AID expression per se was responsible for the low mutation rates in Ramos clone 1, this clone was stably transfected with either a vector expressing human AID (hAID) or an empty vector control. Mutation rates of typical transfected clones were then determined by sequencing unselected V-regions after 1- or 2-months in culture (Table 1). Clones expressing low levels of AID (i.e. clones C.1 and A.1) had very few mutations in the V-region, while clones that expressed ˜25 fold higher levels of AID mRNA (i.e. clones A.2 and A.5) had many more V-region mutations (FIG. 1b and Table 1). Table 2 summarizes the mutational features of all the Ramos clones that expressed elevated levels of AID and shows that the rates and characteristics of the mutations in all of these clones were similar: there was a targeting bias of G/C nucleotides, transitions were slightly favored over transversions and ˜35% of mutations were in RGYW (A/G, G, C/T, A/F) or WRCY hot spot sequences, motifs that are frequently targeted in SHM both in vivo and in vitro (Wagner et al., 1995; Rogozin and Kolchanov, 1992). These data indicate that AID is required for SHM in Ramos cells.

TABLE 1
V-region mutations from cell lines transfected with empty
vector (C) or hAID construct (A)^{a}

| | | | | | Mutation |
|---|---|---|---|---|---|
| | | | | Mutated | rates^{c} |
| | | | Frequency | V- | (mut/ |
| Clone (months | V-region | Total bp | (mut/bp) | regions/ | bp/gen) |
| cultured) | mutations^{b} | sequenced | ×10⁻⁴ | total | ×10⁻⁶ |
| Ramos C.1 (1) | 0 | 11200 | <0.89 | 0/26 | <2.5 |
| Ramos C.1 (2) | 1 | 12900 | 0.78 | 1/30 | 1.1 |
| Ramos A.1 (2)^{d} | 1 | 11600 | 0.86 | 1/27 | 1.2 |
| Ramos A.2 (1) | 5 | 12900 | 3.9 | 4/30 | 10.7 |
| Ramos A.2 (2) | 7 | 12500 | 5.6 | 7/29 | 7.7 |
| Ramos A.5 (1) | 6 | 16300 | 3.7 | 4/38 | 10.2 |
| Ramos A.5 (2) | 13 | 15300 | 8.5 | 9/31 | 11.8 |
| P1-5 C.1 (2) | 1 | 11900 | 0.84 | 1/35 | 1.4 |
| P1-5 C.2 (2) | 0 | 7140 | <1.4 | 0/21 | <2.3 |
| P1-5 A.1 (2) | 28 | 28900 | 9.3 | 22/85 | 15.7 |
| P1-5 A.2 (2) | 6 | 5780 | 10.4 | 6/17 | 17.3 |
| N114 C.1 (1) | 0 | 10980 | <0.91 | 0/18 | <3.0 |
| N114 A.3 (1) | 12 | 21960 | 5.5 | 11/36 | 18.2 |

^{a}Duplicate mutations were counted only once, unless genealogies indicate the mutation was unique.
^{b}The V region corresponds to a 550 bp, 340 bp, and 610 bp region in Ramos, P1-5, and N114 cells, respectively.
^{c}Rates were calculated using a 20-, a 24-, and a 24-hour generation time for Ramos, P1-5, and N114 cells, respectively.
^{d}Expression of AID was low in this clone (FIG. 1b).

Ramos cells express surface markers that suggest that their normal cellular counterpart is a germinal center centroblast (Sale and Neuberger, 1998), which are the cells that normally undergo SHM. Although AID is required for SHM in these cells, other factors specific to centroblasts might also be required. To test this notion, we determined whether AID could induce SHM in hybridomas, which represent plasma cells that are beyond the developmental stage that carries out SHM. We first examined the N89 and N114 hybridomas because they have nonsense codons within the V-region of their endogenous antibody heavy chain gene (Connor et al., 1994; top of FIG. 2a), allowing us to assay many independently transfected clones by assaying for nonsense codon revertants using the ELISA-spot assay (Zhang et al., 2001).

N89 and N114 cells were stably transfected with the hAID expression vector and individual drug-resistant colonies were expanded and assayed for secreted IgM with the ELISA spot assay. Each ELISA spot indicated that a cell had reverted the nonsense codon and was secreting antibody (Lin et al., 1997; FIG. 2a inset). FIG. 2a shows the frequency of revertants identified for each individual clone. None of the N89 and N114 clones that were transfected with the empty vector displayed a revertant frequency above 10⁻⁶ (FIG. 2a). However, more than 50% of individual N89 and N114 clones transfected with the hAID construct had revertant frequencies higher than 10⁻⁶ (FIG. 2a).

To determine the rate of mutation at the nonsense codon of N114, twelve subclones of the AID-positive N114 A.3 clone were analyzed for revertants (FIG. 2a, right panel) yielding a mutation rate of 1.4×10⁻⁶ mut/bp/gen, as calculated by fluctuation analysis (Zhang et al., 2001). As will be shown below, this reversion rate greatly underestimates the mutation rate for the V region as a whole for two reasons: 1) the nonsense codon for N114 (and for N89) is not within an RGYW or WRCY hot spot motif, and 2) most mutations observed in these hybridoma clones were transition mutations in G/C nucleotides (see below) which would convert the TGA nonsense codon to TAA, another nonsense codon.

Some N89 and N114 clones transfected with the hAID expression vector did not revert at a detectable frequency (FIG. 2a). Northern blots revealed that all tested clones that did not express AID did not revert above 10⁻⁶ (FIG. 2b). However, some clones that expressed AID (i.e. N89 A.3, A.5, A.10, A.16, and N114 A.2, A.8; FIG. 2b) also did not revert above 10⁻⁶. While this suggests that AID does not induce SHM in all hybridoma clones, this is probably due to the inherently stochastic nature of this analysis: a nonsense revertant that arises early in the propagation of a clone will result in a culture that has accumulated revertants, while a revertant that arises late during the propagation of the clone will be represented by very few revertants (Luria and Delbrook, 1943). This is exemplified by the high range of revertant frequencies in subclones of N114 A.3, with some subclones having very low rates of reversion (FIG. 2a). This effect is expected to be more pronounced in the AID-transfected N89 clones where mutation rates in the leader sequence (i.e. where the nonsense codon is located) are lower (Rada and Milstein, 2001; Rada et al., 1997).

To measure the real rate of mutation in these AID-expressing hybridomas, transfected clones were grown for 2 months, and unselected V-regions were sequenced. A third hybridoma, P1-5 (Tao and Bothwell, 1990), was included in this analysis because it expresses the same Vh186.2 heavy chain V region that is used by C57BL/6 mice in their response to the hapten nitrophenyl (NP). This allows mutations to be compared between this hybridoma cell and those found in vivo. Only the P1-5 and N114 clones that expressed hAID mutated their V-regions (FIG. 2b, Table 1). In both hybridomas, there were fewer mutations in the constant region than in the V region (1 and 3 mutations for P1-5 and N114, respectively; FIG. 3a), showing that hypermutation was relatively restricted to the V-region. Furthermore, as suggested above, the mutation rates in these hybridoma clones as calculated by sequencing (˜15×10⁻⁶ mut/bp/gen; Table 1) were ˜10 fold higher than those calculated by fluctuation analysis of N114 A.3, as described above (1.4×10⁻⁶ mut/bp/gen; FIG. 2a). These overall mutation rates were similar to those induced by hAID in Ramos clones A.2 and A.5 (i.e. ˜10×10⁻⁶ mut/bp/gen; Table 1). The characteristics of mutations in the N114 hybridoma were also similar to those seen in Ramos cells (Sale and Neuberger, 1998; Table 2). However, the types of mutations found in the V-region of the P1-5 clones were different from the other cell lines in that mutations occurred exclusively in G/C nucleotides, transition mutations occurred at much higher frequencies than transversion mutations, and mutations were mostly found within RGYW and WRCY hot spots (Table 2, FIG. 3b). While this suggests that the mechanism responsible for A/T mutations is absent in the P1-5 hybridoma, which supports the two-phase model of SHM (Rada et al., 1998; Spencer et al., 1999), the high frequency of transition mutations at G/C emphasizes the possibility that AID might be a DNA-specific cytidine deaminase.

TABLE 2
Characteristics of mutations observed in Ramos clones 6 & 7,
and hAID transfected cell lines.

| | | Ramos | | |
|---|---|---|---|---|
| | Ramos clones | clones | P1-5 clones | N114 |
| | 6 & 7^{a} | A.2 & A.5 | A.1 & A.2 | clone A.3 |
| GC | 40/51 (78%) | 25/31 (81%) | 34/34 (100%)^{d} | 9/12 (75%) |
| mutations/ | | | | |
| total | | | | |
| Tₛ^{b}/total | 23/51 (49%) | 13/31 (42%) | 24/34 (71%)^{d} | 7/12 (58%) |
| Deletions/ | 2/51 (4%) | 2/31 (6%) | 0/34 (0%) | 0/12 (0%) |
| total | | | | |
| Hot spot^{c}/ | 18/51 (35%) | 10/31 (32%) | 21/34 (62%)^{d} | 6/12 (50%) |
| total | | | | |

^{a}Previously published sequences (Zhang et al., 2001).
^{b}Tₛ: transitions (C to T or G to A)
^{c}Mutations at underlined nucleotides within RGYW or WRCY are defined as hotspot mutations. 7% (36/550), 9% (32/340), and 6% (39/610) of total nucleotides in V-regions of Ramos, P1-5 and N114, respectively, are hotspot nucleotides.
^{d}Statistically significant when compared to Ramos clones A.2 & A.5 (p < 0.05 for GC mutations, p < 0.001 for Tₛ and for Hot spot mutations).

Discussion

These findings show that AID is sufficient to activate SHM in plasma-like cells, indicating that its activity does not depend on other centroblast-specific factors. We have previously used plasma-like NSO cells to measure SHM of immunoglobulin transgenes that contain V-region nonsense codons (Lin et al., 1997). Sequencing unselected V regions revealed no mutations of the transgenes in 2 month-old cultures indicating low mutation rates in NSO cells. However, fluctuation analysis for nonsense revertants revealed a wide distribution of mutation rates (Id.) which depended strongly on the position of the nonsense codon in the V region, its proximity to hotspot motifs and the number of transgenes present in the transfectant. Of particular interest, we did not detect expression of AID in NSO cells (data not shown). While we cannot rule out transient expression of AID in NSO, it is possible that the high rate of reversion which we observed in a few of the NSO transfectants was engendered by the insertion site and/or the presence of the transgene in a repetitive structure. Nevertheless, we estimate that the overall mutation rates in NSO are low compared to Ramos clones and hybridomas expressing AID. Taken together with recent findings that the pre-B-cell line 18.81 expresses AID and carries out SHM (Bachl et al., 2001), AID might be able to function at any developmental B-cell stage, or perhaps even in non B-cells. This implies that AID functions alone, or with co-factors that are ubiquitously expressed. In fact, many of the factors that have been shown to play a role in SHM, such as MSH2 (Rada et al., 1998; Phung et al., 1998; Reynaud et al., 1999; Vora et al., 1999), MSH6 (Wiesendanger et al., 2000), and DNA polymerases ζ (Zan et al., 2001) and η (Zeng et al., 2001; Rogozin et al., 2001) are enzymes expressed in most cells.

The findings reported here also have practical implications. The fact that hybridomas can be induced to undergo high rates of SHM with expression of AID might allow one to obtain subclones that produce either high-affinity monoclonal antibodies and/or antibodies that are more specific. This has been a goal that has been sought by many (Korbin et al., 1990) since Kohler and Milstein first described the hybridoma technology (Kohler and Milstein, 1975).

### EXAMPLE 2

### AID-Activated Somatic Hypermutation in Non-B-Cells

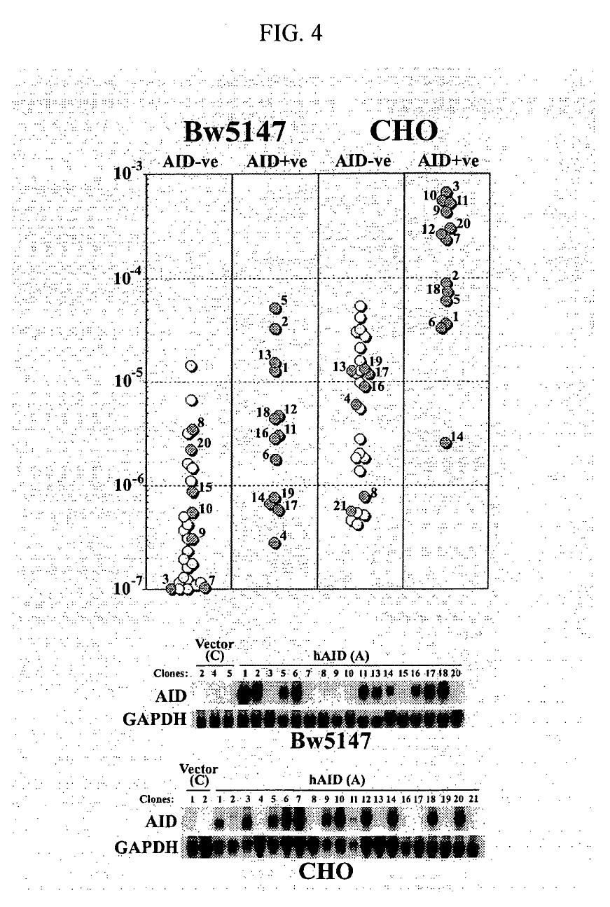

To test whether AID can activate SHM in non-B-cells, Bw5147 (a T-cell line) and CHO (a hamster ovary cell line) were used. These cells were first transfected with immunoglobulin heavy and light chain genes by standard methods. Because the heavy chain gene (Igγ2a) has a nonsense codon in the V-region, the ELISA-spot assay can be used to determine whether AID can turn on SHM in these cells by assessing whether clones have reverted the nonsense codon and secrete IgG2a. Bw5147 and CHO clones that were stably-transfected with the immunoglobulin constructs were then transfected with empty vector or the vector expressing hAID. Individual drug resistant colonies were grown up and assayed by the ELISA-spot assay. FIG. 4 shows that Bw5147 and CHO clones that express hAID have statistically higher reversion frequencies than clones that do not express hAID. These data indicate that hAID can activate SHM in non-B-cells. In addition, because the transacting factors that normally regulate immunoglobulin transcription are not present in these non-B-cells, cis-acting sequences that have been postulated to recruit the mutator to the V-region of the immunoglobulin genes should not be active. This indicates that hAID does not require tissue-specific cis-acting sequences to cause mutations, and further indicates that hAID can mutate any expressed gene.

### EXAMPLE 3

### Induction of Class-Switching by AID

In previous work, forced expression of AID in cultured B-cells (i.e. murine hybridomas and the human Burkitt's lymphoma cell line Ramos) turned on SHM with similar characteristics to that seen in vivo. This suggested that expression of AID was sufficient to initiate this process in B-cells that were at the wrong stage of differentiation. Since AID has also been implicated in class-switch recombination (Kinoshita and Honjo, 2001), we tested whether the Ramos clones that overexpress AID can be induced to class-switch to downstream isotypes. It is worth noting that previous attempts to induce class switch recombination in Ramos has either failed, or been only marginally successful. Since SHM is clonally unstable in Ramos cells (Zhang et al, 2001), and stability is due to the level of AID expression, we reason that Ramos failed to class-switch in those previous experiments because clonally stable Ramos cells were being used that express low levels of AID.

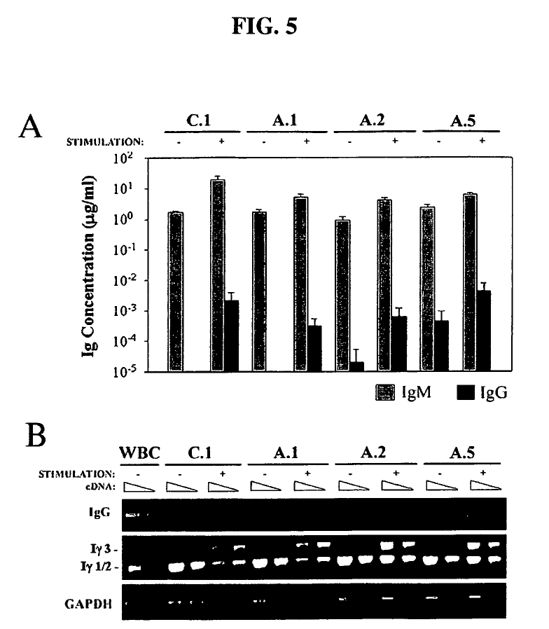

To test this hypothesis, we stimulated the Ramos clones C.1, A.1 (low AID; FIG. 5), and clones A.2, A.5 (high AID; FIG. 5) with CD40L (expressed on NIH3T3 cells) and IL-4. As controls, the same Ramos clones were incubated with NIH3T3 cells that were transfected with empty-vector. As illustrated in FIG. 5A, IgG was detected in the supernatants of all clones, regardless of the level of AID, when stimulated with CD40L and IL-4. However, IgG was also detected in the supernatants of control-stimulated Ramos A.2 and A.5 (FIG. 5A), suggesting that they are hypersensitive to class-switching to the IgG isotype. To confirm these findings, we assessed whether mature IgG message can be identified in unstimulated and stimulated Ramos clones by RT-PCR While IgG mRNA (but not IgA or IgE, data not shown) can be detected in all CD40L and IL-4-stimulated clones, IgG message is only present in Ramos A.2 and A.5 in control stimulations (FIG. 5B). CD40L and IL-4-stimulation causes the production of Iγ3-sterile transcripts in Burkitt's lymphoma cell lines. As shown in FIG. 5B, Iγ3-sterile transcripts are present in all Ramos clones stimulated with CD40L and IL-4, but not in unstimulated clones A.2 and A.5 (FIG. 5B). This suggests that AID functions downstream in the induction of sterile transcripts. Collectively, these data indicate that AID expression hyper-sensitizes Ramos cells to class-switch recombination.

### EXAMPLE 4

### Somatic Hypermutation of the AID Transgene in B and Non-B Cells

This example was published as Martin & Scharff (2002b).

### EXAMPLE SUMMARY

Expression of MD is sufficient to activate SHM in hybridomas, in non-B cells, and in E. coli (See previous examples and Harris et al., 2002), suggesting that it initiates the mutational process by deaminating DNA. However, the cis-acting sequences that are responsible for targeting AID activity to the V-region of immunoglobulin genes are unknown. Here we confirm that expression of AID in B cell lines (i.e. Burkitt's lymphoma Ramos and hybridoma P1-5) not only causes hypermutation of immunoglobulin sequences, but also of other genes, in particular the AID transgene itself. Because it is possible that B cell-specific transacting factors bind to and recruit the ‘mutator’ to the AID transgene, we tested whether the AID transgene can mutate in non-B cells. Indeed, we show that expression of AID in chinese hamster ovary (CHO) cells causes SHM of both the immunoglobulin and AID transgenes. These data confirm that high transcription rates alone appear to predispose any gene to mutation by AID.

Introduction.

Mice and humans with mutations in activation-induced cytidine deaminase (AID) have defects in SHM (Muramatsu et al., 2000; Revy et al 2000), class-switch recombination (Muramatsu et al., 2000), and gene conversion (Arakawa et al., 2000; Harris et al., 2002). Based on the sequence similarity of AID to the RNA-editing enzyme APOBEC-1 (Muramatsu et al., 1999), it was postulated that AID might function by editing a specific message that results in the production of an altered protein that subsequently causes mutation (Kinoshita and Honjo, 2001). However, the higher than expected number of transition mutations in V-regions (Golding et al., 1987) suggests that AID is a DNA-specific cytidine deaminase that converts C to U nucleotides directly in the DNA of the V-region. In fact, we recently showed that AID induced the P1-5 hybridoma to exclusively mutate G•C basepairs and most of these mutations were transition mutations (Martin et al., 2002). With the finding that AID can induce SHM in non-B cells (Yoshikawa et al, 2002) and in E. coli (Harris et al., 2000), it is more likely that AID is a DNA-specific cytidine deaminase.

The mechanism for targeting of SHM to the V region of immunoglobulin genes is not known. SHM has also been observed to occur in other non-immunoglobulin genes. The genes for bcl-6 (Pasqualucci et al., 1998 & 2001; Shen et al., 1998) and FasL (Mushen et al., 2000) undergo SHM with similar characteristics to those observed in the V-region of immunoglobulin genes, albeit at a lower rate. It is possible that these genes share cis-acting sequences with the immunoglobulin locus that are responsible for recruiting AID activity. More likely, B cell-specific cis-acting sequences do not exist, and targeting of SHM to certain genes is due to high transcription rates and possibly other non-specific factors (Martin & Scharff, 2002a). In this regard, since the immunoglobulin gene is one of the most highly transcribed genes in B cells, it would be mutated at higher rates than other genes. However, other transcribed genes have not been found to mutate above the PCR error rate in germinal center B cells (Pasqualucci et al., 2001; Storb et al., 1998a; Shen et al., 2000). Thus, the issue of whether SHM is targeted by B cell-specific cis-acting sequences is not completely resolved. In this report, we confirm SHM of other transcribed genes in B cells and non-B cells activated for SHM by expression of AID.

Materials and Methods.

Constructs: Full length human AID (hAID) cloned into the pCEP4 vector (Invitrogen) has been described before (Martin et al., 2002). The vector was digested with Nru1 and EcoRV prior to transfection. The Vn/ECMV γ2a-construct and the LK-construct were previously described (Lin et al., 1998; Zhu et al., 1995).

Cell lines, cell culture and transfection conditions: Ramos and P1-5 hybridomas were grown as previously described. Chinese hamster ovary (CHO) Pro-5 cells (obtained from P. Stanley, Albert Einstein College of Medicine) were grown in DME medium supplemented with 10% fetal calf serum. CHO cells were first electroporated with 10 μg of the LK-construct. One transfectant that secreted high levels of light-chain (CHO-LC 18) was then transfected with 10 μg of the mouse Vn/ECMV γ2a-construct. One of these transfectants (CHO-LC18-Vn/ECMV clone 8) was then transfected with 10 μg of the linearized hAID or the empty vectors. CHO cells were transfected in DME medium at 400 volts, 960 μF, plated into 96-well plates, and selected with 1.5 mg/ml G418 for the Vn/ECMV γ2a-construct and 0.6 mg/ml hygromycin B for the hAID and empty constructs. The ELISA spot assay was performed as previously reported (Zhang et al., 2001). Briefly, each drug resistant colony was expanded to ˜1-5×10⁶ cells, and plated onto 96-well plates that were pre-coated with anti-mouse IgG2a antibody. After 20 hours, the plates were developed for secreted IgG2a.

PCR Amplification, cloning, and sequencing V-regions and AID transgene: Genomic DNA was prepared as previously reported (Zhang et al., 2001). V-regions from the various B-cell lines were amplified with Pfu polymerase (Stratagene) from genomic DNA using 30 cycles of 95° C./15 sec, 56° C./15 sec, 72° C./30 sec. Primers for AID, 5′ primer: 5′GAGGCAAGAAGACACTCTGG3′, 3′ primer: 5′GTGACATTCCTGGAAGTTGC3′; bcl-6,5′ primer: CCGCTCTTGCCAAATGCTTTG, 3′ primer: CACGATACTTCAT-CTCATCTGG; c-myc, 5′ primer: AGAAAATGGTAGGCGCGCGTA, 3′ primer: TCGACTCATCTCAGCATTAAAG PCR products were cloned and sequenced as previously reported (Zhang et al., 2001). Stratagene reports that PFU polymerase has an error rate of ˜1/650,000 base pairs per duplication. Therefore, in a 30 cycle amplification, we expect ˜1 mutation in 20,000 nucleotides to be attributed to PCR-error. Accession numbers for mutated sequences of the AID gene for Ramos A.2 and A.5 is AF529815-AF529827, for hybridoma P1-5 A.1 and A.2 is AF529828-AF529840, and for CHO A.3 and A.9 is AF529841-AF529856.

Extraction of RNA and Northern Blots: ˜5×10⁶ cells were lysed with 1 ml Trizol reagent (GibcoBRL) and RNA was extracted according to manufacturer instructions. ˜1 μg of total RNA was run on formaldehyde gels for Northern blots.

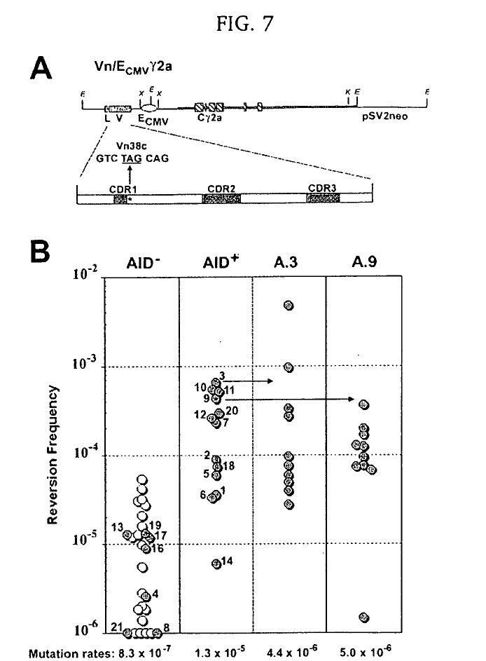

Statistics: Statistics for primary reversion data in FIG. 7B was calculated by the independent-samples t-test with equal variances assumed (SPSS v.10). Results.

We previously showed that the Burkitt's lymphoma Ramos clone 1, which does not undergo SHM and expresses low levels of AID, could be activated for V-region SHM by overexpressing AID (Martin et al., 2002). Specifically, the V-region (V) accumulates many mutations in Ramos clones A.2 and A.5 (Table 3) that have ˜25 fold higher levels of AID mRNA than Ramos clones C.1 and A.1 (Id.). Bcl-6 and the c-myc allele that has translocated into the switch region have also been shown to undergo SHM in B cell lines (Bemark and Neuberger, 2000; Zan et al., 2000). To determine whether overexpression of AID in Ramos cells also activated mutation in these genes, bcl-6 and c-myc were sequenced from two month-old cultures of Ramos clones that overexpressed AID. However, only a few mutations were found in the c-myc and bcl-6 genes in Ramos clones A.2 and A.5 (data not shown). RT-PCR analysis revealed that these genes were indeed being transcribed (data not shown). Because SHM correlates with transcription rates (Bachl et al., 2001), it is possible that the rates of transcription of these genes might be too low for SHM to occur at the level seen in the V-region.

TABLE 3
Somatic hypermutation of the V region and of the AID transgene
in Ramos, hybridoma P1-5, and CHO cells.

| | Level of | | | | | |
|---|---|---|---|---|---|---|
| | AID | | | | | Mutation |
| Clone | expression¹ | | | Frequency | Mutated | rates³ |
| (months | (vector | | Total bp | (mut/bp) | sequences/ | (mut/bp/gen) |
| cultured) | used) | Mutations² | sequenced | ×10⁻⁴ | total | ×10⁻⁶ |
| Ramos | AID^{low} | 1 (V)⁴ | 12900 | 0.78 | 1/30 | 1.1 |
| C.1 (2) | (empty | | | | | |
| | vector) | | | | | |
| Ramos | AID^{low} | 1 (V)⁴ | 11600 | 0.86 | 1/27 | 1.2 |
| A.1 (2) | (sense | | | | | |
| | hAID) | | | | | |
| Ramos | AID^{hi} | 7 (V)⁴ | 12500 | 5.6 | 7/29 | 7.7 |
| A.2 (2) | (sense | 7 (A) | 17400 | 4.0 | 6/29 | 5.6 |
| | hAID) | | | | | |
| Ramos | AID^{hi} | 13 (V)⁴ | 15300 | 8.5 | 9/31 | 11.8 |
| A.5 (2) | (sense | 8 (A) | 15600 | 5.1 | 7/26 | 6.9 |
| | hAID) | | | | | |
| Ramos | AID^{low} | 2 (A) | 15020 | 1.3 | 2/29 | 3.6 |
| αA.1 (1) | (anti-sense | | | | | |
| | hAID) | | | | | |
| Ramos | AID^{low} | 1 (A) | 8200 | 1.2 | 1/14 | 3.3 |
| αA.2 (1) | (anti-sense | | | | | |
| | hAID) | | | | | |
| P1-5 A.1 | AID^{hi} | 28 (V)⁴ | 28900 | 9.3 | 22/85 | 15.7 |
| (2) | (sense | 10 (A) | 14400 | 6.9 | 9/24 | 11.6 |
| | hAID) | | | | | |
| P1-5 A.2 | AID^{hi} | 6 (V)⁴ | 5780 | 10.4 | 6/17 | 17.3 |
| (2) | (sense | 7 (A) | 6600 | 10.6 | 5/11 | 17.7 |
| | hAID) | | | | | |
| CHO | AID^{hi} | 6 (A) | 12600 | 4.8 | 6/21 | 8.0 |
| A.3 (2) | (sense | | | | | |
| | hAID) | | | | | |
| CHO | AID^{hi} | 11 (A) | 11400 | 9.6 | 11/19 | 16.0 |
| A.9 (2) | (sense | | | | | |
| | hAID) | | | | | |
| CHO | AID^{neg} | 1 (A) | 10800 | 0.9 | 1/18 | 3.0 |
| αA.1 (1) | (anti-sense | | | | | |
| | hAID) | | | | | |
| CHO | AID^{neg} | 1 (A) | 9490 | 1.1 | 1/16 | 3.7 |
| αA.5 (1) | (anti-sense | | | | | |
| | hAID) | | | | | |

¹Expression levels of AID for Ramos clones was previously published (8). Expression of AID was negative (AID^{neg}), low (AID^{low}), or high (AID^{hi}).
²Mutations identified in the V-region (V) = 430 bp, AID transgene (A) = 600 bp.
³Mutation rates were calculated using a 20-, a 24-, and a 24-hour generation time for Ramos, P1-5, and CHO cells, respectively.
⁴Data previously published (8) from the identical clones and DNA samples used to analyze AID mutations.

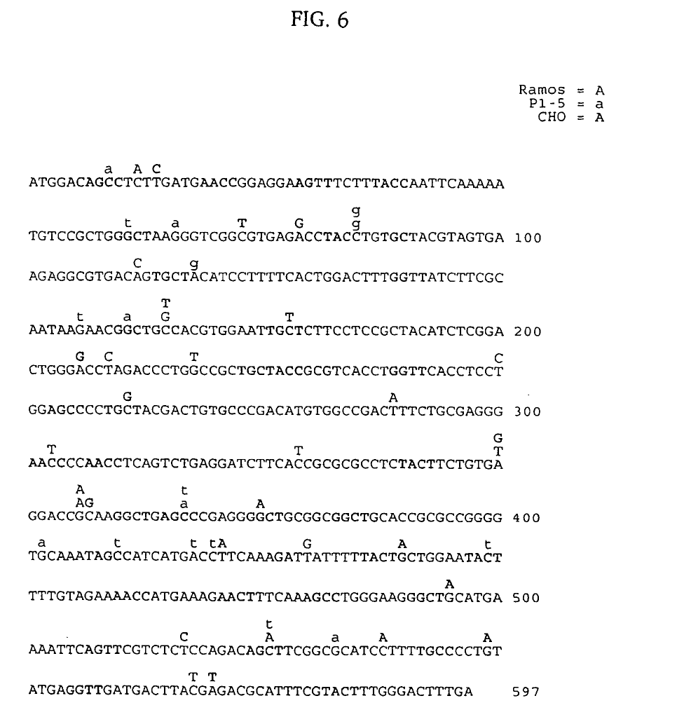

To test whether a highly transcribed non-immunoglobulin gene was undergoing SHM in the same Ramos clones, we sequenced the highly transcribed AID transgene that is regulated by the CMV promoter. Indeed, many mutations were identified within the AID transgene (A) in Ramos clones A.2 and A.5 (FIG. 6 and Table 3), and the calculated rates of mutation were only slightly lower than that of the V-region (Tables 3 and 4). In addition, the characteristics of the mutations in the AID transgene were similar to those in the V region (Table 4). In particular, 20% of all mutations occurred at G•C basepairs within RGYW or WRCY hot spot motifs, even though only 7% of G/C nucleotides within the AID transgene occur at these sequences (Table 4). RGYW and WRCY hotspot motifs are frequently mutated during SHM in vitro and in vivo (Rogozin and Kolchanov, 1992). To confirm that mutations observed in the AID transgene were due to the AID protein, the non-mutating Ramos clone 1 was transfected with a construct in which the AID transgene is in the antisense orientation to transcription. In this case, only a few mutations were found within the AID transgene (Ramos clones αA.1 and αA.2: (A); Table 3). To confirm that the hAID construct that was used for transfection did not contain mutations, AID was amplified from the hAID plasmid with PFU polymerase, cloned, and sequenced. Only one mutation was found at an A•T basepair in 10 clones (6000 nucleotides) sequenced.

TABLE 4
Characteristics of mutations observed in the V region and the AID
transgene in Ramos, P1-5, and CHO cells.

| | Ramos | | P1-5 | | CHO |
|---|---|---|---|---|---|
| | | AID | | AID | AID |
| | V region | transgene | | transgene | transgene |
| | A.2 & | A.2 & | V region | A.1 & | A.3 & |
| Characteristic | A.5¹ | A.5 | A.1 & A.2¹ | A.2 | A.9 |
| Mutation | 9.8 | 6.3 | 16.5 | 14.7 | 12.0 |
| rates² | | | | | |
| GC | 25/31 (81%) | 8/15 (53%) | 34/34 (100%) | 15/17 (88%) | 11/17 (65%) |
| mutations/total | | | | | |
| Tₛ³ mutations/ | 13/31 (42%) | 8/15 (53%) | 24/34 (71%) | 10/17 (59%) | 11/17 (65%) |
| total | | | | | |
| Hot spot⁴/total | 10/31 (32%) | 3/15 (20%) | 21/34 (62%) | 8/17 (47%) | 4/17 (24%) |

¹Data previously published (8).
²Mut/bp/gen.
¹Data previously published (8).
²Mut/bp/gen.
³Transition mutation (i.e. C to T, T to C, G to A, A to G)
⁴G·C basepairs within RGYW/WRCY motifs are designated as hotspot nucleotides. 39/597 (7%) of nucleotides in AID transgene, and 7% (36/550) and 9% (32/340) of nucleotides in the V region of Ramos and P1-5, respectively, are hot spot nucleotides.

To determine if SHM of the AID transgene can also occur in other B cell lines, we tested whether the AID transgene was being mutated in the P1-5 hybridoma. This hybridoma is unique in that AID expression induces mutations exclusively at G•C basepairs in the endogenous V region that are mostly within RGYW/WRCY hotspot motifs (P1-5 clones A.1 and A.2; Table 4)(Martin et al., 2002). This suggests that this cell line is missing a factor(s) responsible for the A•T mutations that are believed to occur during the second phase of SHM that is MMR- and polymerase η-dependent (Rogozin et al., 2001; Zeng et al., 2001; Rada et al., 1998). Sequencing of the AID transgene in 2-month old cultures revealed many mutations (FIG. 6 & Table 3). The calculated rates and frequencies of mutation in the endogenous V region and in the ectopically-integrated AID transgene were similar (Table 3 and 4), and a striking bias for mutations in RGYW/WRCY motifs was observed in both genes: 62% and 47% of all mutations were within RGYW/WRCY motifs in the V-region and the AID transgene, respectively (Table 4). In addition, like in the V-region, most mutations occurred at G•C basepairs. The few A•T mutations in the AID transgene may have arisen by a non-AID related processes, such as during the integration of the transgene into the genome (Wilkie and Palmiter, 1987). These data indicate that AID mutates both itself and the immunoglobulin gene in B cell lines.

The data presented above suggest that hypermutation induced by AID does not require specific cis-acting sequences to localize mutation to a specific gene. This is because the AID transgene is not expected to share regulatory sequences with the immunoglobulin loci. However, it is formally possible that the hAID transgene and the CMV promoter-enhancer in particular contain sequences similar to the immunoglobulin loci that are required for targeting SHM. It is also possible that the sites of integration that allow high expression of AID contain regulatory sequences that share motifs with the antibody gene. Because non-B cells are not considered to have B cell-specific transacting factors, expressing the AID transgene in a non-B cell should cause any putative B cell-specific cis-acting sequence to be silent. Thus, mutation of the AID transgene in non-B cells would argue against the requirement of regulatory B cell-specific cis-acting sequences for targeting SHM. We therefore tested whether the AID transgene can mutate in non-B cells.

To confirm that human AID can activate SHM in chinese hamster ovary (CHO) cells, CHO cells were first stably transfected with murine heavy and light chain immunoglobulin genes (FIG. 7A). The murine heavy chain construct used in this experiment (i.e. Vn/ECMV γ2a-construct; FIG. 7A) has two unique features. First, the intronic μ enhancer was replaced with the CMV enhancer to ensure that the immunoglobulin heavy chain gene was expressed in CHO cells. Northern blots confirm that the Vn/ECMV γ2a-transgene was expressed (data not shown). Second, a nonsense codon was introduced into an RGYW hotspot in the variable region of the heavy chain construct (FIG. 7A). This allows SHM to be measured by reversion of the nonsense codon that would result in the production and secretion of IgG2a that could be detected at the single cell level using the ELISA-spot assay.

CHO cells stably expressing the murine immunoglobulin genes were transfected with the hAID transgene, the antisense hAID transgene, and the empty vector control. Independent transfectants were grown to approximately 2×10⁶ cells, and distributed into ELISA plates coated with anti-murine γ2a antibody. After 20 hours, the ELISA plates were developed for secreted antibody. The revertant frequency for each individual clone was then plotted (FIG. 7B, left panels). Because some hAID-transfected CHO cells did not express hAID (i.e. CHO clones A.4, A.8, A.13, A.16, A.17, A.19, A.21, FIG. 4), the revertant frequencies for each individual CHO clone was plotted in the relevant AID-negative (AID−) and AID-positive (AID+) columns (FIG. 7B). As shown in FIG. 7B (left panels), clones that express hAID reverted the nonsense-codon in the Vn/ECMV γ2a-transgene ˜15 fold more frequently than clones that did not express AID (p<0.01). To more accurately determine the mutation rates at the nonsense codon, 2 AID+ clones (i.e. CHO A.3 and A.9) were subcloned. Ten subclones of each were assayed by the ELISA spot assay, and mutation rates were calculated by fluctuation analyses (19). CHO clones A.3 and A.9 displayed mutation rates of 4.4×10⁻⁶ and 5.0×10⁻⁶ mutations per base pair per generation (mut/bp/gen), respectively (FIG. 7B, right panels). Although these two clones chosen for further analysis initially reverted at high frequencies (FIG. 7B, left panels), the corresponding subclones displayed a similar range of reversion frequencies and mutation rates to that of the larger group of independently transfected AID+CHO clones. It is unclear why CHO clones that do not express AID have such a high background of reversion frequencies (AID−; FIG. 7B, left panel). Nevertheless, these data support findings (Yoshikawa et al., 2002) that AID can induce SHM in non-B cells.

To test whether the AID transgene was mutating in CHO cells, AID was sequenced from 2 month-old cultures of CHO clones A.3 and A.9. The AID transgene was found to contain many mutations in the sense (CHO clones A.3 and A.9; FIG. 6 and Table 3) but not in the antisense orientation (CHO clones αA.1 and αA.5; Table 3). The calculated rates of mutation of the AID transgene were similar between the CHO clones and the B cell lines (Table 4), and the characteristics of the mutations displayed a similar pattern typical to SHM in cultured cells, namely a bias towards mutations in G-C basepairs, a preference for transition mutations, and RGYW hot spot targeting (Table 4).

Discussion.

The work reported here indicates that the SHM process is not dependent on a specific cis-acting sequence(s) to target mutation to the immunoglobulin gene, and will proceed with any cis-acting sequence that confers a high rate of transcription to the target gene. Two of our findings support this hypothesis: I) an immunoglobulin transgene mutates in a non-B cell, and 2) the AID transgene driven by a strong promoter mutates in B and non-B cells. Although it is possible that the AD transgene has a B cell-specific cis-acting sequence(s), if this sequence element were to exist, it should be inactive in non-B cells since non-B cells lack B cell-specific transacting factors. Similar findings to those reported here were described in fibroblasts activated to mutate a substrate when AID was overexpressed (Yoshikawa et al., 2002). The lack of a requirement of specific cis-acting sequences is also supported by findings that other genes undergo SHM in B cells (Pasqualucci et al., 1998 & 2001; Shen et al., 1998; Muschen et al., 2000), and other genes essential for cell viability might also be mutated since constitutive SHM appears to decrease the viability of cultured cells (Zhang et al., 2001). While the notion that SHM can occur in any highly transcribed gene is unsettling, this may explain why many types of lymphomas arise from B cells that are undergoing SHM (Pasqualucci et al., 2001; Kuppers and Dalla-Favera, 2001).

On the other hand, some observations support the notion that SHM is regulated by cis-acting sequences. First, other transcribed genes in germinal center B cells do not undergo SHM (Pasqualucci et al., 2001; Storb et al., 1998a, Shen et al., 2000). The critical issue here is whether these genes are in fact mutating, but at levels that are below the PCR error rate, or whether they are not mutating at all. Because mutation rates are positively correlated with transcription rates (Bachl et al., 2001) and with RGYW/WRCY hot spot density (Michael et al., 2002), other genes might in fact be mutating, but at rates that simply correlate with the quantity of these other features. In this regard, the immunoglobulin gene might be mutated at a higher rate than other genes because it is transcribed at very high rates. In addition, it must also be considered that accumulation of mutations downstream of promoters will only occur in regions that do not confer a selective disadvantage, such as regions that do not contain open reading frames or regulatory sequences for housekeeping genes. Mutations in these regions should reduce the viability of the cell, and as a consequence, the apparent rate of mutation at these loci will seem to be low or absent.

Second, the classical observation that the V-region mutates at higher rates than the C-region also supports the idea that cis-acting sequences are involved in targeting SHM. For example, a B cell-specific cis-acting sequence might affect the chromatin structure over the V-region allowing the ‘mutator’ to gain access to the DNA. However, other explanations exist that could account for this differential mutation of the V- versus C-regions without the requirement of B cell-specific cis-acting sequences. One possibility is that the ‘mutator’ associates with the RNA polymerase II complex to produce mutations during the initiation phase, but eventually falls off this complex during the elongation phase (Maizels, 1995; Storb et al., 1998b). Another possibility is that mutation depends on the availability of single-stranded DNA (see below), and that there is more single-stranded DNA in the V-region than in the C-region. This in turn might be due to 1) stable RNA-DNA hybrids in the V-region as a result of transcription that leaves the non-transcribed strand single-stranded, 2) a higher RNA polymerase II density in the V-region than in the C-region, or 3) transcription inducing stable secondary DNA structures in the V-region with single-stranded loops of DNA (Kinoshita and Honjo, 2001). While these models suggest that mutation can be focused on the V-region without the requirement of cis-acting sequences, there is presently no data to support these beliefs.

Many of the findings that support the notion that SHM is not regulated by B cell-specific cis-acting sequences come from reports, including this one, where AID is overexpressed at levels that are believed to be higher than in centroblasts (Martin et al., 2002; Yoshikawa et al., 2002; Okawaki et al., 2002), although this value is not known. Because overexpression of APOBEC-1, which is homologous to AID, resulted in hyper-editing of its target substrate ApoB mRNA (Davidson and Shelness, 2000), it is possible that targeting of SHM has been deregulated in cells that overexpress AID. Thus, caution must be exercised when interpreting this data. In addition, mutation rates induced by expression of AID in cell lines are ˜10 fold lower than the rates observed in the V-region in vivo (Martin and Scharff 2002a). Thus other B cell-specific factors might indeed help focus mutation over the V-region.

AID has been postulated to function directly (i.e. to directly deaminate cytidines in DNA [Kinoshita & Honjo, 2001; Martin et al., 2002]) and indirectly (i.e. via its putative mRNA editing activity [Kinoshita & Honjo, 2001]) in the SHM process. The fact that human AID can activate SHM in a hamster ovary cell line and in E. coli (Petersen-Mahrt et al., 2002) argues against AID having an indirect role in SHM since this would require that the transcript edited by AID be expressed ubiquitously and have the same recognition motif in different species. Further support to the notion that AID is a DNA-specific cytidine deaminase was provided by the finding that AID predominantly caused transition mutations at G-C basepairs in the P1-5 hybridoma (Martin et al., 2002), in fibroblasts (Yoshikawa et al., 2002), and in E. coli (Petersen-Mahrt, 2002). In addition, the mutation rates induced by AID in E. coli is increased slightly in the absence of uracil DNA glycosylase (Petersen-Mahrt et al., 2002) suggesting that uracil is an intermediate in the SHM process. Thus, AID might initiate SHM by deaminating cytidines on DNA resulting in the recruitment of the mismatch repair system and/or uracil DNA glycosylases (Martin and Scharff, 2002a; Petersen-Mahrt, 2002; Poltoratsky et al., 2000), which in turn could cause mutations at other basepairs during the repair phase. Since enzyme-catalyzed cytidine deamination probably requires single-stranded DNA (because the amino group on cytidine is hydrogen-bonded to the carboxyl of guanosine), AID might therefore chose its target based on the availability of single-stranded DNA (Martin and Scharff, 2002a).

The findings presented in this report also have practical implications. Because the AID transgene was found to mutate, it is likely that any transgene under the regulation of a strong promoter will mutate as long as AID is expressed in that cell. Semi-random mutagenesis can facilitate the characterization of the gene products for structure/function analysis.

### EXAMPLE 5

### Flanking a Gene with Foreign Sequences Inhibits Somatic Hypermutation for that Gene

As shown in Example 4, the ectopically-integrated CMV-driven AID transgene (with a hygromycin-resistance selectable marker) undergoes somatic hypermutation in B and non-B cells. That is, AID mutates both the immunoglobulin genes and itself. However, when a second vector (with a puromycin-resistance selectable marker), which had the CMV-driven AID transgene flanked by ˜2 kb vector-derived bacterial sequences, was integrated into N114 hybridoma cells, the AID gene did not undergo somatic hypermutation, even though immunoglobulin genes did mutate with this AID transgene. This lack of somatic hypermutation of the AID transgene was not due to reduced transcription of the AID gene in the cells transfected with the second vector, because the AID transgene was transcribed at the same rates in cells transfected with either vector. Thus the difference in mutation between the two vectors is not due to transcription differences, but is apparently due to the presence of flanking bacterial sequences in the second vector.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method of inducing a mutation in a gene in a eukaryotic cell, wherein the gene is operably linked to a promoter, and wherein the gene is within about two kilobases of the promoter, the method comprising expressing a transgenic activation-induced cytidine deaminase (AID) gene in the cell.

2. The method of claim 1, wherein the gene is also operably linked to an enhancer.

3. The method of claim 2, wherein the enhancer is an immunoglobulin enhancer.

4. The method of claim 1, wherein the gene is between 10 bases and 2 kb in the 3′ direction from the promoter.

5. The method of claim 1, wherein the promoter is an immunoglobulin promoter.

6. The method of claim 1, wherein a polyA mRNA of the gene is synthesized in the cell, the polyA mRNA of the gene comprising at least 0.01% of total polyA mRNA in the cell.

7. The method of claim 6, wherein the polyA mRNA of the gene comprises at least 0.1% of total polyA mRNA in the cell.

8. The method of claim 6, wherein the polyA mRNA of the gene comprises at least 0.5% of total polyA mRNA in the cell.

9. The method of claim 6, wherein the polyA mRNA of the gene comprises at least 1% of total polyA mRNA in the cell.

10-12. (canceled)

13. The method of claim 1, wherein the AID gene is flanked by a sequence foreign to the cell, wherein the sequence foreign to the cell is at least 200 bp long.

14. (canceled)

15. The method of claim 13, wherein the sequence foreign to the cell is at least 2000 bp long.

16-17. (canceled)

18. The method of claim 1, wherein the cell is a yeast cell.

19. The method of claim 1, wherein the cell is a vertebrate cell.

20. The method of claim 19, wherein the cell is a mammalian cell.

21. The method of claim 20, wherein the cell is a B-cell.

22. The method of claim 20, wherein the cell is a hybridoma.

23. The method of claim 20, wherein the cell is a human cell.

24. The method of claim 1, wherein the gene is an antibody gene.

25. The method of claim 1, wherein the gene encodes a protein selected from the group consisting of an enzyme, a transcription factor, a cytokine, and a structural protein.

26-33. (canceled)

34. A method of determining the effect of mutations in a gene encoding a protein on the phenotype of the protein in a eukaryotic cell, wherein the gene is operably linked to a promoter, and wherein the gene is within about two kilobases of the promoter, the method comprising
(a) expressing the protein and a transgenic AID gene in the eukaryotic cell;
(b) establishing clonal colonies of the cell;
(c) identifying clonal colonies that produce a gene of the protein that has a mutation;
(d) determining whether the protein expressed by the mutated gene in any clonal colony identified in step (c) has an altered phenotype; and
(e) associating the altered phenotype with a particular mutation.

35-57. (canceled)

58. A method of inducing a mutation in an antibody gene in a eukaryotic cell, the method comprising expressing a transgenic AID gene in the cell.

59-96. (canceled)

97. A method of inducing a class switch in an antibody heavy chain gene in a eukaryotic cell, the method comprising expressing a transgenic AID gene in the cell.

98-124. (canceled)

125. A method of altering an affinity or a specificity of a monoclonal antibody to an antigen, or altering a cross-reactivity of the monoclonal antibody to a second antigen, wherein the monoclonal antibody is produced by a eukaryotic cell, and wherein the cell is capable of expressing a transgenic AID gene under inducible control, the method comprising
(a) expressing the AID gene in the eukaryotic cell for a time and under conditions sufficient to induce a mutation in a gene encoding the monoclonal antibody;
(b) suppressing expression of AID gene in the eukaryotic cell;
(c) establishing clonal colonies of the cell; and
(d) determining whether the monoclonal antibody produced by any of the clonal colonies of the cell has altered affinity or specificity to the antigen, or altered cross-reactivity to the second antigen.

126-158. (canceled)

159. A eukaryotic cell comprising a transgenic AID gene, wherein expression of the AID gene is inducible.

160-171. (canceled)

172. The cell of claim 159, wherein the cell is a myeloma cell.

173. The cell of claim 159, wherein the cell is a hybridoma cell.

174-179. (canceled)

180. A eukaryotic cell expressing an AID gene, wherein the cell is not a B-cell.

181-190. (canceled)

191. The cell of claim 180, wherein the cell is a yeast cell.

192. The cell of claim 180, wherein the cell is a vertebrate cell.

193. The cell of claim 192, wherein the cell is a mammalian cell.

194. The cell of claim 193, wherein the cell is a human cell.

195-202. (canceled)

203. A myeloma fusion partner expressing an AID gene.

204-212. (canceled)

213. The myeloma fusion partner of claim 203, wherein the fusion partner is selected from the group consisting of a Sp2/0-Ag 14, a FOX-NY, a P3X63, NX-1, a P3, a P3X643 Ag8.653, a NS1, and a NSO.

214. A hybridoma expressing an AID gene.

215-261. (canceled)
